(19) United States
(12) Patent Application Publication
(10) Pub. No.: US 2024/0293403 A1
(43) Pub. Date: Sep. 5, 2024

(54) Treatment of Symptoms Associated with Myeloproliferative Neoplasms

(71) Applicant: Telios Pharma, Inc., Redwood City, CA (US)

(72) Inventor: Wayne Rothbaum, New York, NY (US)

(73) Assignee: Telios Pharma, Inc., Redwood City, CA (US)

(21) Appl. No.: 18/570,297

(22) PCT Filed: Jun. 16, 2022
(86) PCT No.: PCT/US2022/033728

§ 371 Date: Dec. 14, 2023

Related U.S. Application Data
(60) Provisional application No. 63/211,276, filed on Jun. 16, 2021.

(51) Int. Cl. A61K 31/506; A61K 31/497; A61K 31/519; A61K 31/5377; A61K 45/06

(52) U.S. Cl. CPC A61K 31/506; A61K 31/497; A61K 31/519; A61K 31/5377; A61K 45/06

(57) ABSTRACT

Therapeutic methods and pharmaceutical compositions for treating symptoms associated with myeloproliferative neo-plasms in a human subject are described. In certain embodiments, the disclosure includes therapeutic methods of treating symptoms associated with myeloproliferative neoplasms using a BTK inhibitor.

### FIELD OF THE DISCLOSURE

Methods of treating symptoms associated with myeloproliferative neoplasms using a Bruton's Tyrosine Kinase (BTK) inhibitor are disclosed herein.

### BACKGROUND

Myeloproliferative neoplasms (MPNs) are blood cancers that occur when the body makes too many white blood cells, red blood cells, and/or platelets. MPNs include essential thrombocythemia (ET), polycythemia vera (PV) and myelofibrosis (MF), and can lead to significant rates of morbidity and mortality among afflicted patients.

MF, for example, is a chronic leukemia, a cancer that affects the blood-forming tissues in the body. MF is an uncommon type of bone marrow cancer that disrupts the normal production of blood cells. MF causes extensive scarring in bone marrow, leading to severe anemia that can cause weakness and fatigue and it can also cause a low number of platelets, which increases the risk of bleeding. MF often causes an enlarged spleen and lymph nodes due to the accumulation of CD34+ malignant myeloid cells in the spleen.

The clinical spectrum of MF includes primary myelofibrosis and MF that develops during essential thrombocythemia or polycythemia vera. Myelofibrosis is a chronic hematologic malignancy characterized by splenomegaly, leukoerythroblastosis, cytopenias, teardrop poikilocytosis, marrow fibrosis, extramedullary hematopoiesis, increased marrow microvessel density, and constitutive mobilization of hematopoietic stem cells (HSC) and progenitor cells (HPC) that express CD34. Ruxolitinib, fedratinib and allogeneic stem cell transplantation are the primary means of treating patients with MF.

In general, symptomatic burden in MPN is severe and has a significant impact on patient quality of life. Complications of MPNs include constitutional symptoms related to aberrant overproduction of pro-inflammatory cytokines (e.g., fatigue, night sweats, weight loss, pruritus, fever, bone and joint pain) and symptoms associated with splenomegaly. Methods of reducing or alleviating these symptoms in patients suffering from MPNs are needed to improve patients' quality of life.

Bruton's Tyrosine Kinase is a non-receptor tyrosine kinase that belongs to the Tec family and has an important function in several benign and malignant cells of the hematopoietic system. Moreover, recent clinical studies with irreversible oral BTK inhibitors, acalabrutinib and ibrutinib, have demonstrated excellent clinical activity and tolerability against a variety of B-cell malignancies including: chronic lymphocytic leukemia (CLL), mantle cell lymphoma (MCL), Waldenstrom macroglobulinemia (WM), marginal zone lymphoma (MZL) and diffuse large B-cell lymphoma (DLBCL). Furthermore, it is now clear the mechanism of action of BTK inhibitors is multifactorial, with a significant component of its function in lymphoid malignancies involving the disruption of the tumor cell and the microenvironment that protects it. Inhibition of BTK has been shown to regulate CLL, MCL and malignant myeloid cell migration in acute myeloid leukemia by inhibiting CXCR4-CXCL12 induced cell trafficking, homing and integrin adhesion by downregulating expression of numerous vascular adhesion molecules (Zaitseva (2014) Oncotarget 5, 9930-9938). CXCL12 plays a central role in CLL pathogenesis and progression, by regulating CLL cell interaction with the stromal microenvironment, leading to cell survival and proliferation. BTK has a role in signal transduction activated by the CXCR4-CXCL12 signaling axis and is involved in rapid integrin activation. BTK inhibition prevents CXCL12-induced triggering of lymphocyte function-associated antigen-1 (LFA-1) and VLA-4 integrins. Furthermore, BTK inhibition blocks the activation of the small GTP-binding protein RhoA, controlling integrin affinity. Very importantly, BTK tyr-phosphorylation and activation by CXCL12 depends on upstream activation of JAK2 (Janus kinase 2). Thus, BTK and JAK protein tyrosine kinases manifest a hierarchical activity both in chemokine and integrin activation and dependent cell adhesion (Montresor (2018) Oncotarget, 9, 35123-35140). Lastly, BTK is highly expressed on both mature and primitive myeloid cells; including HSC and HPC.

### SUMMARY OF THE DISCLOSURE

The disclosure relates to a method of reducing or alleviating a symptom selected from the group consisting of night sweats, fatigue, pruritus, abdominal pain, pain under ribs, fullness, bone pain, and combinations thereof in a human subject with a myeloproliferative neoplasm (MPN) comprising administering to the human subject an amount of a Bruton's Tyrosine Kinase (BTK) inhibitor effective to reduce or alleviate the symptom. In some embodiments, the MPN is primary myelofibrosis. In some embodiments, the MPN is post-polycythemia vera myelofibrosis. In some embodiments, the MPN is post-essential thrombocythemia myelofibrosis. In some embodiments, the MPN is chronic myelogenous leukemia. In some embodiments, the MPN is chronic neutrophilic leukemia. In some embodiments, the MPN is chronic eosinophilic leukemia. In some embodiments, the MPN is acute myelogenous leukemia with antecedent MPN (also known as blast phase MPN (MPN-BP)). In some embodiments, the MPN is chronic myelomonocytic leukemia. In some embodiments, the human subject is suffering from splenomegaly, hepatomegaly, or hepatosplenomegaly.

In some embodiments, the BTK inhibitor is selected from Table 1 of the specification or a pharmaceutically acceptable salt thereof. In some embodiments, the BTK inhibitor is 1-(4-(((6-amino-5-(4-phenoxyphenyl)pyrimidin-4-yl)amino)methyl)-4-fluoropiperidin-1-yl)prop-2-en-1-one or a pharmaceutically acceptable salt thereof.

In some embodiments, the BTK inhibitor is administered in combination with a JAK2 inhibitor. In some embodiments, the JAK2 inhibitor is ruxolitinib, fedratinib, pacritinib, momelotinib, jaktinib, or ilginatinib. In some embodiments, the BTK inhibitor is administered in combination with a MDM2 inhibitor.

In some embodiments, the human subject has a JAK2V617F mutation. In some embodiments, the human subject is homozygous for the JAK2V617F mutation. In some embodiments, the MPN is a JAK2-V617F myeloproliferative neoplasm.

In some embodiments, the symptom persists for at least one day prior to administration of the BTK inhibitor, such as at least two days, at least three days, at least four days, at least five days, at least six days, or at least seven days prior to administration of the BTK inhibitor.

In some embodiments, the method further comprises assessing the severity of the symptom prior to administration of the BTK inhibitor. In some embodiments, the method further comprises assessing the severity of the symptom after administration of the BTK inhibitor. In some embodiments, severity of the symptom is determined by self-assessment. In some embodiments, severity of the symptom is determined by self-reporting.

In some embodiments, the night sweats comprise repeated episodes of perspiration during sleep of the human subject. In some embodiments, the night sweats comprise repeated episodes of extreme perspiration during sleep of the human subject. In some embodiments, the fatigue comprises unexplained and relapsing exhaustion of the human subject. In some embodiments, the fatigue comprises unexplained, persistent, and relapsing exhaustion of the human subject. In some embodiments, the pruritus comprises itching of the skin of the human subject. In some embodiments, the pruritus comprises severe itching of the skin of the human subject. In some embodiments, the abdominal pain occurs between the chest and pelvic regions and is acute. In some embodiments, the abdominal pain occurs between the chest and pelvic regions and is chronic. In some embodiments, the pain under ribs occurs on the left side of the human subject and is acute. In some embodiments, the pain under ribs occurs on the left side of the human subject and is chronic. In some embodiments, the fullness is associated with satiety in the human subject. In some embodiments, the bone pain is characterized by tenderness, aching, or other discomfort in one or more bones of the human subject. In some embodiments, the bone pain is characterized by extreme tenderness, aching, or other discomfort in one or more bones of the human subject.

### DETAILED DESCRIPTION

While preferred embodiments of the disclosure are shown and described herein, such embodiments are provided by way of example only and are not intended to otherwise limit the scope of the disclosure. Various alternatives to the described embodiments may be employed in practicing the disclosure.

Unless defined otherwise, all technical and scientific terms used herein have the same meaning as is commonly understood by one of skill in the art to which this disclosure belongs.

The terms “administered in combination with” and “co-administration” as used herein, encompass administration of two or more active pharmaceutical ingredients to a subject so that both agents and/or their metabolites are present in the subject at the same time. Co-administration includes simultaneous administration in separate compositions, administration at different times in separate compositions, or administration in a composition in which two or more agents are present.

The term “amount effective” or “effective amount” or “therapeutically effective amount” or “amount sufficient” refers to that amount of an active pharmaceutical ingredient or combination of active pharmaceutical ingredients as described herein that is sufficient to effect the intended application including, but not limited to, disease treatment, reducing a symptom, or alleviating a symptom. A therapeutically effective amount may vary depending upon the intended application (in vitro or in vivo), or the subject and disease condition being treated (e.g., the weight, age and gender of the subject), the severity of the disease condition, the manner of administration, and other factors which can readily be determined by one of ordinary skill in the art. The term also applies to a dose that will induce a particular response in target cells (e.g., malignant CD34+ myeloid cells). The specific dose will vary depending on the particular compounds chosen, the dosing regimen to be followed, whether the compound is administered in combination with other compounds, timing of administration, the tissue to which it is administered, and the physical delivery system in which the compound is carried.

“Myelofibrosis” refers to spontaneous scarring (fibrosis) of the bone marrow that disrupts the normal production of blood cells, leading to severe anemia and enlargement of the spleen, lymph nodes and liver. It can be associated with a variety of diseases, primarily myeloproliferative (preleukemic) disorders. It is also known as agnogenic myeloid metaplasia. Myelofibrosis, as used herein, includes but is not limited to, primary myelofibrosis, post-polycythemia vera myelofibrosis, and post-essential thrombocythemia myelofibrosis. Myelofibrosis as used herein, is characterized by accumulation of malignant CD34+ myeloid cells in the bone marrow, spleen and lymph nodes.

“Pharmaceutically acceptable carrier” or “pharmaceutically acceptable excipient” is intended to include any and all solvents, dispersion media, coatings, antibacterial and antifungal agents, isotonic, and absorption delaying agents. The use of such media and agents for active pharmaceutical ingredients is well known in the art. Except insofar as any conventional media or agent is incompatible with the active pharmaceutical ingredient, its use in the therapeutic compositions of the disclosure is contemplated. Supplementary active ingredients can also be incorporated into the described compositions.

“Solvate” refers to a compound in physical association with one or more molecules of a pharmaceutically acceptable solvent.

The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In selected embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve proton transfer between the cocrystal and the drug, and instead involves intermolecular interactions, such as hydrogen bonding, aromatic ring stacking, or dispersive forces, between the cocrystal former and the drug in the crystal structure.

The terms “QD,” “qd,” or “q.d.” means quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “splenomegaly” as used herein refers to an enlargement of the spleen, measured by size or weight, and typically measured by volume determined by medical imaging. In some embodiments, the enlargement is due to sequestration of malignant CD34+ myeloid cells and the resulting extramedullary hemopoiesis which develops.

A “therapeutic effect” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

BTK inhibitor compounds of the disclosure also include crystalline and amorphous forms of the any of the compounds in Table 1, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### Methods of Reducing or Alleviating Symptoms of Myeloproliferative Neoplasms (MPNs)

The present disclosure relates to the discovery that a BTK inhibitor can be used to reduce or alleviate various symptoms of MPNs including, for example, night sweats, fatigue, pruritus, abdominal pain, pain under ribs, fullness, bone pain, and combinations thereof. Accordingly, in certain aspects, the disclosure relates to methods for reducing or alleviating a symptom selected from night sweats, fatigue, pruritus, abdominal pain, pain under ribs, fullness, bone pain, or a combination thereof by administering to a human subject in need thereof an effective amount of a BTK inhibitor, optionally in combination of one or more other supportive therapies or active agents for treating MPNs. The disclosure also relates to methods for reducing or alleviating a symptom selected from night sweats, fatigue, pruritus, abdominal pain, pain under ribs, fullness, bone pain, or a combination thereof by administering to a human subject with an MPN an effective amount of a BTK inhibitor, optionally in combination of one or more other supportive therapies or active agents for treating MPNs. The disclosure herein demonstrates that desirable therapeutic agents may be selected on the basis of BTK inhibition. Therefore, while not wishing to be bound to a particular mechanism of action, it is expected that BTK inhibition alters one or more downstream signaling components (e.g., CXCR-4, CXCL12, VLA-4, VCAM-1) to mobilize migration of CD34+ cells into the peripheral blood where they will be useful in reducing or alleviating symptoms associated with MPNs, particularly in reducing or alleviating symptoms including, but not limited to, night sweats, fatigue, pruritus, abdominal pain, pain under ribs, fullness, bone pain, and combinations thereof.

The present disclosure thus relates to a method of reducing or alleviating symptoms associated with MPNs in a human subject suffering from an MPN comprising the step of administering to the human a BTK inhibitor. In some embodiments, the MPN is myelofibrosis (MF). In some embodiments, the MF is primary myelofibrosis, also known as chronic idiopathic myelofibrosis (cIMF). In an embodiment, the primary myelofibrosis is selected from the group consisting of prefibrotic/early stage PMF and overt fibrotic stage PMF. In some embodiments, the MF is secondary myelofibrosis. In some embodiments, the MF is myelofibrosis that develops secondary to polycythemia vera or essential thrombocythemia. In some embodiments, the MF is myelofibrosis that develops secondary to chronic myeloid leukemia (CML). In some embodiments, the MF is idiopathic myelofibrosis. In some embodiments, the MPN is chronic myelogenous leukemia. In some embodiments, the MPN is acute myelogenous leukemia with antecedent MPN (also known as blast phase MPN (MPN-BP)). In some embodiments, the MPN is a chronic neutrophilic leukemia (CNL), a chronic eosinophilic leukemia, or a chronic myelomonocytic leukemia (CMML). In some embodiments, the MPN is an idiopathic systemic myeloproliferative neoplasm (MPN-U), a myelodysplastic syndrome (MDS), a systemic mast cell disease (SMCD), an atypical chronic myeloid leukemia (aCML), a juvenile myelomonocytic leukemia (JMML), a hypereosinophilic syndromes (HES), and a myelodysplastic/myeloproliferative neoplasm with ring sideroblasts and thrombocytosis (MDS/MPN-RS-T).

In an embodiment, the human is determined as hydroxyurea (HU) intolerant (unacceptable side effects). In an embodiment, the human subject is determined as hydroxyurea resistant (inadequate response).

In some embodiments, the human subject is additionally suffering from splenomegaly, hepatomegaly, or hepatosplenomegaly. In an embodiment, the human subject has splenomegaly, hepatomegaly, or hepatosplenomegaly and is phlebotomy-dependent. In an embodiment, the human subject is phlebotomy-dependent without splenomegaly, hepatomegaly, or hepatosplenomegaly.

In an embodiment, the human subject has elevated levels of pro-inflammatory cytokines and administration of the BTK inhibitor reduces the elevated pro-inflammatory cytokines. In an embodiment, the pro-inflammatory cytokine is selected from interleukin-1 (IL-1), interleukin-2 (IL-2), interleukin-6 (IL-6), interleukin-8 (IL-8), interleukin-12 (IL-12), interleukin-18 (IL-18), tumor necrosis factor alpha (TNF-α), interferon gamma (IFNγ), granulocyte-macrophage colony stimulating factor (GM-CSF), granulocyte colony stimulating factor (G-CSF), hepatocyte growth factor (HGF), platelet-derived growth factor (PDGF), epidermal growth factor (EGF), vascular endothelial growth factor (VEGF), or a combination thereof. In an embodiment, administration of the BTK inhibitor to the human subject downregulates nuclear factor-kappa B (NF-κB). In an embodiment, administration of the BTK inhibitor to the human subject upregulates anti-inflammatory cytokines. In an embodiment, the anti-inflammatory cytokine is selected from interleukin-4 (IL-4), interleukin-10 (IL-10), or a combination thereof. In an embodiment, the human subject has splenomegaly, hepatomegaly, or hepatosplenomegaly, and administration of the BTK inhibitor to the human subject ameliorates the splenomegaly, hepatomegaly, or hepatosplenomegaly.

In an embodiment, the BTK inhibitor is any of the compounds in Table 1 or a pharmaceutically acceptable salt thereof.

In an embodiment, the human subject is JAK2 inhibitor naïve (i.e. has never received therapy with a JAK2 inhibitor. In an embodiment, the human subject is JAK2 inhibitor intolerant. In an embodiment, the human subject is JAK2 inhibitor ineligible due to a low platelet count. In an embodiment, the human subject has relapsed after JAK2 inhibitor treatment. In an embodiment, the human subject is refractory to JAK2 inhibitor treatment. In an embodiment, the human subject failed ruxolitinib or fedratinib therapy. Failed ruxolitinib or fedratinib therapy includes, but is not limited to, (i) the absence of a reduction in the severity or progression of any myeloproliferative neoplasm in a human subject receiving ruxolitinib or fedratinib, or (ii) a relapse of any myelofibrosis in a human subject following ruxolitinb or fedratinib therapy. In an embodiment, failed ruxolitinib or fedratinib therapy is the absence of a reduction in the severity or progression of any myelofibrosis in a human subject receiving ruxolitinib or fedratinib. In an embodiment, failed ruxolitinib or fedratinib therapy is a relapse of any myelofibrosis in a human subject following ruxolitinb or fedratinib therapy.

In an embodiment, the BTK inhibitor is administered in combination with a JAK2 inhibitor. In an embodiment, the JAK2 inhibitor is ruxolitinib, fedratinib, pacritinib, momelotinib, jaktinib, ilginatinib, or a combination thereof.

In an embodiment, the BTK inhibitor is administered in combination with a MDM2 inhibitor.

In an embodiment, the MPN is a JAK2-V617F myeloproliferative neoplasm.

In an embodiment, the human subject has an accumulation of malignant CD34+ myeloid cells in their spleen. In an embodiment, the malignant CD34+ myeloid cells have decreased expression of CXCR4 relative to normal myeloid cells. In an embodiment, administration of the BTK inhibitor stimulates migration of the malignant CD34+ myeloid cells from the spleen to the peripheral blood of the human subject. In an embodiment, administration of the BTK inhibitor inactivates VLA-4 in the malignant CD34+ myeloid cells. In an embodiment, administration of the BTK inhibitor reduces activity of CXCR4 and CXCL12 thereby reducing the chemoattract effects these molecules on malignant CD34+ myeloid cells. In an embodiment, reduction of CXCR4 and CXCL12 activity contributes to the sequestration of malignant myeloid CD34+ cells in the spleen.

In an embodiment, the method encompasses reducing or alleviating a symptom associated with myelofibrosis such as night sweats, fatigue, pruritus, abdominal pain, pain under ribs, fullness, bone pain, and combinations thereof, but not treating myelofibrosis itself (e.g. only the symptoms of myelofibrosis are reduced or alleviated in the human and not the myelofibrosis).

In an embodiment, the BTK inhibitor is administered in a dosage selected from the group consisting of 15 mg QD, 25 mg QD, 30 mg QD, 50 mg QD, 60 mg QD, 75 mg QD, 90 mg QD, 100 mg QD, 120 mg QD, 150 mg QD, 175 mg QD, 180 mg QD, 200 mg QD, 225 mg QD, 240 mg QD, 250 mg QD, 275 mg QD, 300 mg QD, 325 mg QD, 350 mg QD, 360 mg QD, 375 mg QD, 480 mg QD, 560 mg QD, 15 mg BID, 25 mg BID, 30 mg BID, 50 mg BID, 60 mg BID, 75 mg BID, 90 mg BID, 100 mg BID, 120 mg BID, 150 mg BID, 175 mg BID, 180 mg BID, 200 mg BID, 225 mg BID, 240 mg BID, 250 mg BID, 275 mg BID, 300 mg BID, 325 mg BID, 350 mg BID, 360 mg BID, 375 mg BID, and 480 mg BID. In an embodiment, the BTK inhibitor is administered to a human according to the Section herein titled Dosages and Dosing Regimens.

In an embodiment, the human suffering from a symptom such as night sweats, fatigue, pruritus, abdominal pain, pain under ribs, fullness, bone pain, and combinations thereof has myelofibrosis characterized by the presence of a CALR mutation (calreticulin, located on chromosome 19p13.2) in the human subject as described in Massie, New Engl. J. Med. (2013) 25, 2379-2390 and incorporated by reference herein in its entirety.

In an embodiment, the human suffering from a symptom such as night sweats, fatigue, pruritus, abdominal pain, pain under ribs, fullness, bone pain, and combinations thereof has myelofibrosis characterized by the presence of an MPL mutation (myeloproliferative leukemia virus oncogene; located on chromosome 1p34) in the human subject as described in Pikman, Plos Med. (2006) 3, e270 and incorporated by reference herein in its entirety.

In an embodiment, the human suffering from a symptom such as night sweats, fatigue, pruritus, abdominal pain, pain under ribs, fullness, bone pain, and combinations thereof has myelofibrosis characterized by JAK2V617F mutation in the human subject. JAK2V617F is a function mutation promoting cytokine-independent growth of myeloid cells, as described in Nakatake (Oncogene (2012) 31, 1323-1333) and incorporated by reference herein in its entirety.

In an embodiment, the human suffering from a symptom such as night sweats, fatigue, pruritus, abdominal pain, pain under ribs, fullness, bone pain, and combinations thereof has myelofibrosis characterized by one or more mutations selected from the group consisting of JAK2V617F, MPL, CALR and combinations thereof.

In an embodiment, the human suffering from a symptom such as night sweats, fatigue, pruritus, abdominal pain, pain under ribs, fullness, bone pain, and combinations thereof has myelofibrosis characterized by the absence of the JAK2V617F mutation, the absence of the MPL mutation, the absence of the CALR mutation, the absence of a combination of two of the JAK2V617F mutation, the MPL mutation, and the CALR mutation, or the absence of all three of the JAK2V617F mutation, the MPL mutation, and the CALR mutation. In an embodiment, the human suffering from a symptom such as night sweats, fatigue, pruritus, abdominal pain, pain under ribs, fullness, bone pain, and combinations thereof has myelofibrosis characterized by the absence of the JAK2V617F mutation.

TABLE 1
BTK Inhibitors

| No. | IUPAC Name | Structure |
|---|---|---|
| 1. | Acalabrutinib ((S)-4-(8-amino-3-(1-(but- 2-ynoyl)pyrrolidin-2-yl)imidazo[1,5- a]pyrazin-1-yl)-N-(pyridin-2- yl)benzamide) | 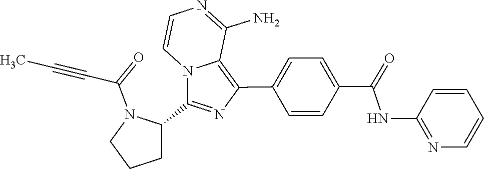 [SMILES: CC#CC(=O)N1CCC[C@H]1c1nc(-c2ccc(C(=O)Nc3ccccn3)cc2)c2c(N)nccn12] |
| 2. | Ibrutinib (1-[(3R)-3-[4-amino-3-(4- phenoxyphenyl)pyrazolo[3,4- d]pyrimidin-1-yl]piperidin-1-yl]prop-2- en-1-one | 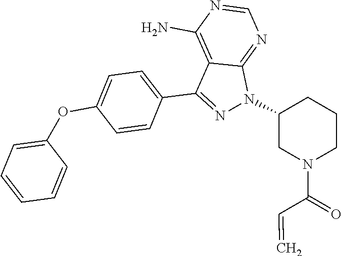 [SMILES: C=CC(=O)N1CCC[C@@H](n2nc(-c3ccc(Oc4ccccc4)cc3)c3c(N)ncnc32)C1] |
| 3. | (7S)-2-(4-phenoxyphenyl)-7-(1-prop-2- enoylpiperidin-4-yl)-4,5,6,7- tetrahydropyrazolo[1,5-a]pyrimidine-3- carboxamide | 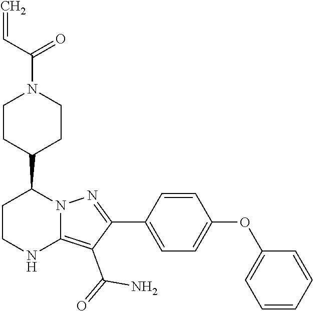 [SMILES: C=CC(=O)N1CCC([C@@H]2CCNc3c(C(N)=O)c(-c4ccc(Oc5ccccc5)cc4)nn32)CC1] |
| 4. | 2-(4-phenoxyphenyl)-7-(1-prop-2- enoylpiperidin-4-yl)-4,5,6,7- tetrahydropyrazolo[1,5-a] pyrimidine-3-carboxamide | 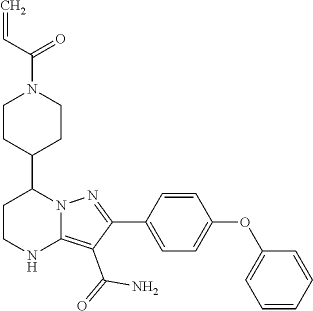 [SMILES: C=CC(=O)N1CCC(C2CCNc3c(C(N)=O)c(-c4ccc(Oc5ccccc5)cc4)nn32)CC1] |
| 5. | (7R)-2-(4-phenoxyphenyl)-7-(1-prop-2- enoylpiperidin-4-yl)-4,5,6,7- tetrahydropyrazolo[1,5-a]pyrimidine-3- carboxamide | 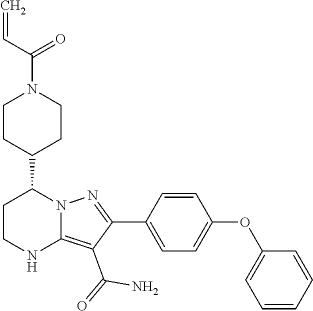 [SMILES: C=CC(=O)N1CCC([C@H]2CCNc3c(C(N)=O)c(-c4ccc(Oc5ccccc5)cc4)nn32)CC1] |
| 6. | 6-amino-9-[(3R)-1-but-2- ynoylpyrrolidin-3-yl]-7-(4- phenoxyphenyl)purin-8-one | 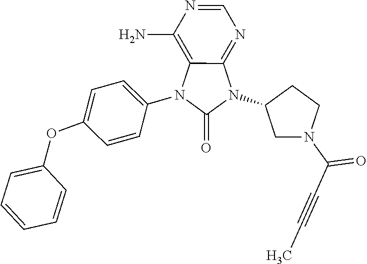 [SMILES: CC#CC(=O)N1CC[C@@H](n2c(=O)n(-c3ccc(Oc4ccccc4)cc3)c3c(N)ncnc32)C1] |
| 7. | N-[3-[[5-fluoro-2-[4-(2- methoxyethoxy)anilino]pyrimidin-4- yl]amino]phenyl]prop-2-enamide | 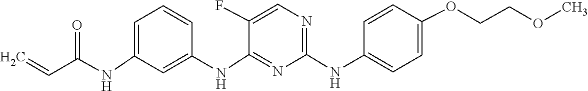 [SMILES: C=CC(=O)Nc1cccc(Nc2nc(Nc3ccc(OCCOC)cc3)ncc2F)c1] |
| 8. | Fenebrutinib (10-[3-(hydroxymethyl)-4- [1-methyl-5-[[5-[(2S)-2-methyl-4- (oxetan-3-yl)piperazin-1-yl]pyridin-2- yl]amino]-6-oxopyridin-3-yl]pyridin-2- yl]-4,4-dimethyl-10- diazatricyclo[6.4.0.0^{2,6}]dodeca-2(6),7- dien-9-one | 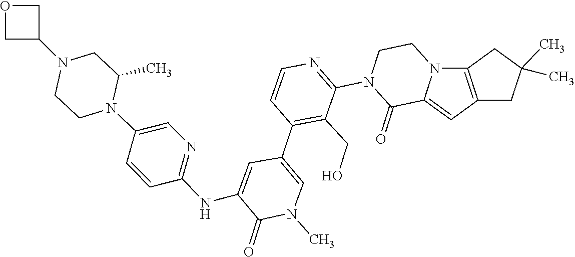 [SMILES: C[C@H]1CN(C2COC2)CCN1c1ccc(Nc2cc(-c3ccnc(N4CCn5c(cc6c5CC(C)(C)C6)C4=O)c3CO)cn(C)c2=O)nc1] |
| 9. | 1-[4-[[[6-amino-5-(4- phenoxyphenyl)pyrimidin-4- yl]amino]methyl]piperidin-1-yl]prop-2- en-1-one | 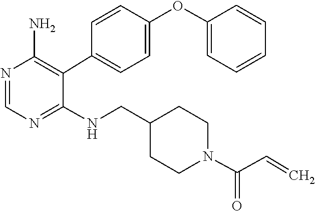 [SMILES: C=CC(=O)N1CCC(CNc2ncnc(N)c2-c2ccc(Oc3ccccc3)cc2)CC1] |
| 10. | 1-[4-[[[6-amino-5-(4- phenoxyphenyl)pyrimidin-4- yl]amino]methyl]piperidin-1-yl]prop-2- en-1-one | 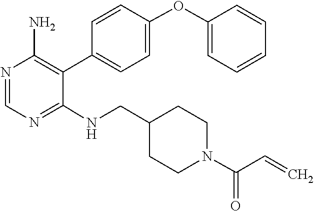 [SMILES: C=CC(=O)N1CCC(CNc2ncnc(N)c2-c2ccc(Oc3ccccc3)cc2)CC1] |
| 11. | (2-chloro-4-phenoxyphenyl)-[4- [[(3R,6S)-6-(hydroxymethyl)oxan-3- yl]amino]-7H-pyrrolo[2,3-d]pyrimidin- 5-yl]methanone | 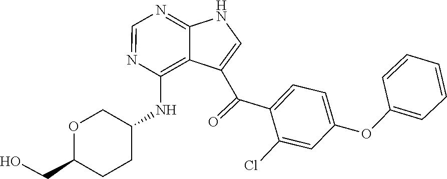 [SMILES: O=C(c1ccc(Oc2ccccc2)cc1Cl)c1c[nH]c2ncnc(N[C@@H]3CC[C@@H](CO)OC3)c12] |
| 12. | N-[3-[6-[4-[(2R)-1,4-dimethyl-3- oxopiperazin-2-yl]anilino]-4-methyl-5- oxopyrazin-2-yl]-2-methylphenyl]- 4,5,6,7-tetrahydro-1-benzothiophene- 2-carboxamide | 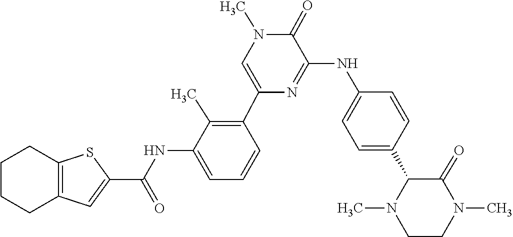 [SMILES: Cc1c(NC(=O)c2cc3c(s2)CCCC3)cccc1-c1cn(C)c(=O)c(Nc2ccc([C@@H]3C(=O)N(C)CCN3C)cc2)n1] |
| 13. | 2-[2-[2-[4-[4-amino-3-(4- phenoxyphenyl)pyrazolo[3,4- d]pyrimidin-1-yl]piperidin-1- yl]ethoxy]ethoxy]-N-[2-(2,6- dioxopiperidin-3-yl)-1,3- dioxoisoindol-5-yl]acetamide | 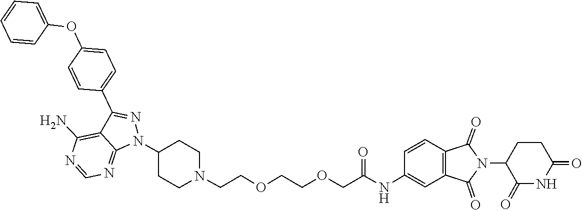 [SMILES: Nc1ncnc2c1c(-c1ccc(Oc3ccccc3)cc1)nn2C1CCN(CCOCCOCC(=O)Nc2ccc3c(c2)C(=O)N(C2CCC(=O)NC2=O)C3=O)CC1] |
| 14. | N-[3-[2-[4-(4-methylpiperazin-1- yl)anilino]furo[3,2-d]pyrimidin-4- yl]oxyphenyl]prop-2-enamide | 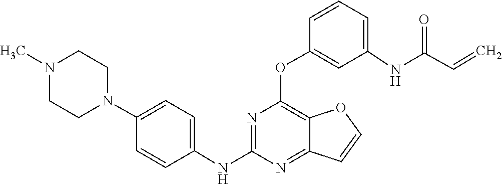 [SMILES: C=CC(=O)Nc1cccc(Oc2nc(Nc3ccc(N4CCN(C)CC4)cc3)nc3ccoc23)c1] |
| 15. | 4-tert-butyl-N-[2-methyl-3-[1-methyl-5- [4-(morpholine-4-carbonyl)-3-(prop- 2-enoylamino)anilino]-6- oxopyridin-3-yl]phenyl]benzamide | 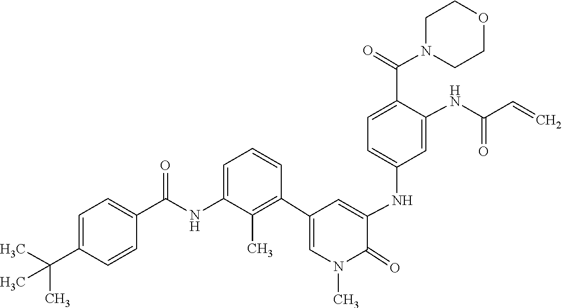 [SMILES: C=CC(=O)Nc1cc(Nc2cc(-c3cccc(NC(=O)c4ccc(C(C)(C)C)cc4)c3C)cn(C)c2=O)ccc1C(=O)N1CCOCC1] |
| 16. | (R,E)-2-(3-(4-amino-3-(2-fluoro-4- phenoxyphenyl)-1H-pyrazolo[3,4- d]pyrimidin-1-yl)piperidine-1- carbonyl)-4-methyl-4-(4- (oxetan-3-yl)piperazin-1- yl)pent-2-enenitrile | 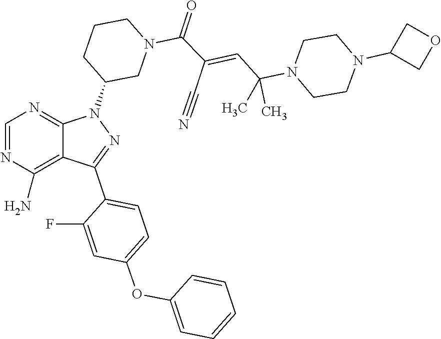 [SMILES: CC(C)(/C=C(\C#N)C(=O)N1CCC[C@@H](n2nc(-c3ccc(Oc4ccccc4)cc3F)c3c(N)ncnc32)C1)N1CCN(C2COC2)CC1] |
| 17. | Branebrutinib ((S)-4-(3-(but-2- ynamido)piperidin-1-yl)-5-fluoro-2,3- dimethyl-1H-indole-7-carboxamide) | 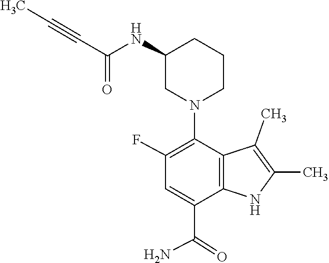 [SMILES: CC#CC(=O)N[C@H]1CCCN(c2c(F)cc(C(N)=O)c3[nH]c(C)c(C)c23)C1] |
| 18. | 4-(tert-Butyl)-N-(2-methyl-3-(4-methyl- 6-((4-(morpholine-4- carbonyl)phenyl)amino)-5-oxo-4,5- dihydropyrazin-2-yl)phenyl)benzamide | 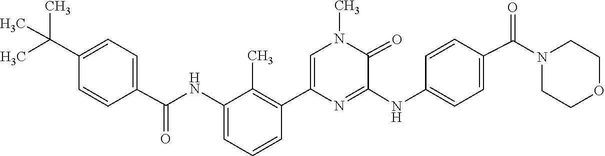 [SMILES: Cc1c(NC(=O)c2ccc(C(C)(C)C)cc2)cccc1-c1cn(C)c(=O)c(Nc2ccc(C(=O)N3CCOCC3)cc2)n1] |
| 19. | N-(1-(7H-Pyrrolo[2,3-d]pyrimidin-4- yl)piperidin-3-yl)-2-((3- chlorophenyl)amino)acetamide | 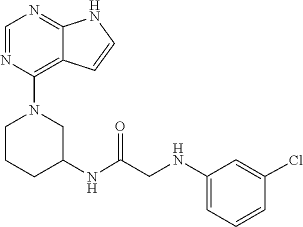 [SMILES: O=C(CNc1cccc(Cl)c1)NC1CCCN(c2ncnc3[nH]ccc23)C1] |
| 20. | 6-cyclopropyl-8-fluoro-2-[2- (hydroxymethyl)-3-[1-methyl-5-[[5-(4- methylpiperazin-1-yl)pyridin-2- yl]amino]-6-oxopyridin-3- yl]phenyl]isoquinolin-1-one | 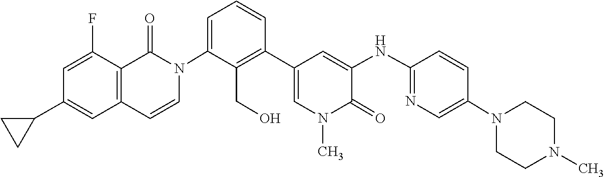 [SMILES: CN1CCN(c2ccc(Nc3cc(-c4cccc(-n5ccc6cc(C7CC7)cc(F)c6c5=O)c4CO)cn(C)c3=O)nc2)CC1] |
| 21. | N-[5-[9-[4- (methanesulfonamido)phenyl]-2- oxobenzo[h][1,6]naphthyridin-1-yl]-2- methylphenyl]prop-2-enamide | 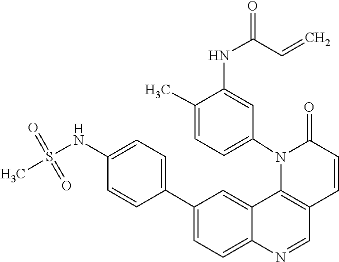 |
| 22. | 4-(4-((4-((3-acrylamidophenyl)amino)-5- fluoropyrimidin-2-yl)amino)phenoxy)-N- methylpicolinamide | 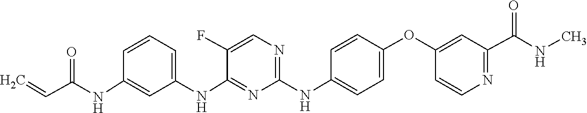 [SMILES: C=CC(=O)Nc1cccc(Nc2nc(Nc3ccc(Oc4ccnc(C(=O)NC)c4)cc3)ncc2F)c1] |
| 23. | (7S)-3-fluoro-4-[3-(8-fluoro-1-methyl- 2,4-dioxoquinazolin-3-yl)-2- methylphenyl]-7-(2-hydroxypropan-2- yl)-6,7,8,9-tetrahydro-5H-carbazole-1- carboxamide | 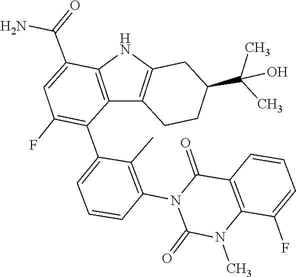 [SMILES: Cc1c(-c2c(F)cc(C(N)=O)c3[nH]c4c(c23)CC[C@H](C(C)(C)O)C4)cccc1-n1c(=O)c2cccc(F)c2n(C)c1=O] |
| 24. | 1-[3-fluoro-4-[7-(5-methyl-1H-imidazol- 2-yl)-1-oxo-2,3-dihydroisoindol-4- yl)phenyl]-3-[3- (trifluoromethyl)phenyl]urea | 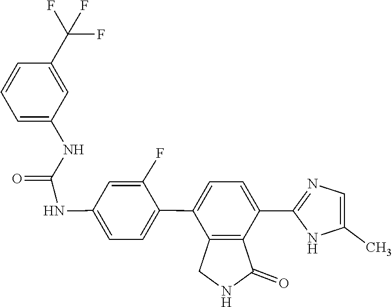 [SMILES: Cc1cnc(-c2ccc(-c3ccc(NC(=O)Nc4cccc(C(F)(F)F)c4)cc3F)c3c2C(=O)NC3)[nH]1] |
| 25. | 9-(1-methylpyrazol-4-yl)-1-(1-prop-2- enoy-2,3-dihydroindol-6- yl)benzo[h][1,6]naphthyridin-2-one | 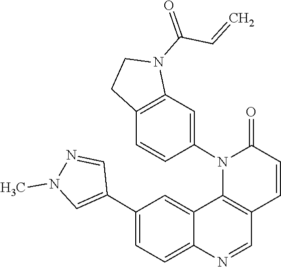 [SMILES: C=CC(=O)N1CCc2ccc(-n3c(=O)ccc4cnc5ccc(-c6cnn(C)c6)cc5c43)cc21] |
| 26. | 7-(2-hydroxypropan-2-yl)-4-[2-methyl- 3-(4-oxoquinazolin-3-yl)phenyl]-9H- carbazole-1-carboxamide | 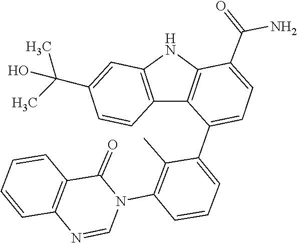 [SMILES: Cc1c(-c2ccc(C(N)=O)c3[nH]c4cc(C(C)(C)O)ccc4c23)cccc1-n1cnc2ccccc2c1=O] |
| 27. | 10[2-(Hydroxymethyl)-3-[1-methyl-6- oxo-5-(pyrimidin-4-ylamino)pyridin-3- yl]phenyl]-4,4-dimethyl-7-thia-10- azatricyclo[6.4.0.02,6]dodeca-1(8),2(6), dien-9-one | 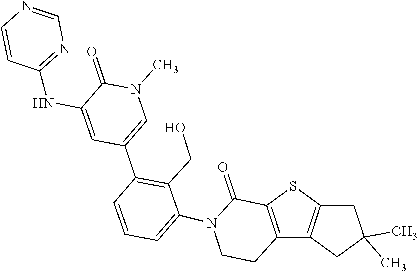 [SMILES: Cn1cc(-c2cccc(N3CCc4c(sc5c4CC(C)(C)C5)C3=O)c2CO)cc(Nc2ccncn2)c1=O] |
| 28. | (S)-5-amino-1-(1-cyanopiperidin-3-yl)-3- (4-(2,4-difluorophenoxy)phenyl)-1H- pyrazole-4-carboxamide | 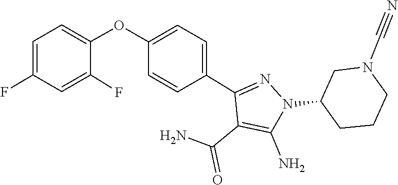 [SMILES: N#CN1CCC[C@H](n2nc(-c3ccc(Oc4ccc(F)cc4F)cc3)c(C(N)=O)c2N)C1] |
| 29. | (S)-4-(3-(1-Acryloylpyrrolidin-2-yl)-8- aminoimidazo[1,5-a]pyrazin-1-yl)-N- (pyridin-2-yl)benzamide | 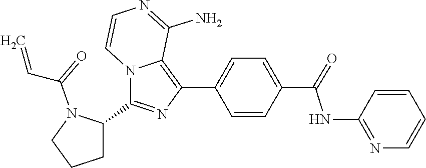 [SMILES: C=CC(=O)N1CCC[C@H]1c1nc(-c2ccc(C(=O)Nc3ccccn3)cc2)c2c(N)nccn12] |
| 30. | (S,E)-4-(8-Amino-3-(1-(4- (dimethylamino)but-2-enoyl)pyrrolidin- 2-yl)imidazo[1,5-a]pyrazin-1-yl)- N(pyridin-2-yl)benzamide | 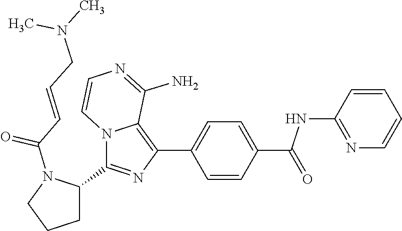 [SMILES: CN(C)C/C=C/C(=O)N1CCC[C@H]1c1nc(-c2ccc(C(=O)Nc3ccccn3)cc2)c2c(N)nccn12] |
| 31. | (S)-4-(8-Amino-3-(1-but-2- ynoylpyrrolidin-2-yl)imidazo[1,5- a]pyrazin-1-yl)-N-(4-methylpyridin-2- yl)benzamide | 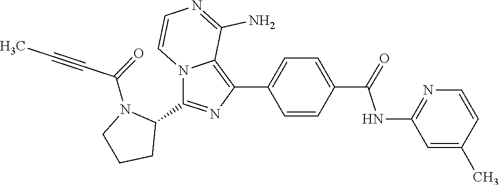 [SMILES: CC#CC(=O)N1CCC[C@H]1c1nc(-c2ccc(C(=O)Nc3cc(C)ccn3)cc2)c2c(N)nccn12] |
| 32. | (S,E)-4-(8-Amino-3-(1-(4-methoxybut-2- enoyl)pyrrolidin-2-yl)imidazo[1,5- a]pyrazin-1-yl)-N-(4-propyl- pyridin-2-yl)benzamide | 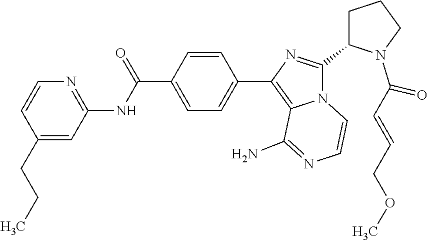 [SMILES: CCCc1ccnc(NC(=O)c2ccc(-c3nc([C@@H]4CCCN4C(=O)/C=C/COC)n4ccnc(N)c34)cc2)c1] |
| 33. | (S)-4-(8-Amino-3-(1-but-2- ynoylpyrrolidin-2-yl)imidazo[1,5-a] pyrazin-1-yl)-N-(4- (trifluoromethyl)pyridin-2- yl)benzamide | 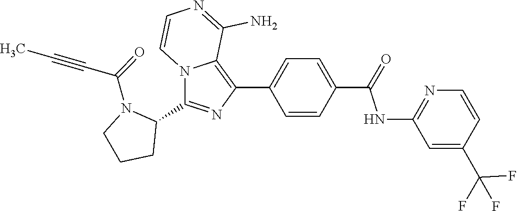 [SMILES: CC#CC(=O)N1CCC[C@H]1c1nc(-c2ccc(C(=O)Nc3cc(C(F)(F)F)ccn3)cc2)c2c(N)nccn12] |
| 34. | (S)-4-(8-Amino-3-(1-but-2- ynoylpyrrolidin-2-yl)imidazo[1,5- a]pyrazin-1-yl)-N-(4,5,6,7- tetrahydrobenzo[d]thiazol-2- yl)benzamide | 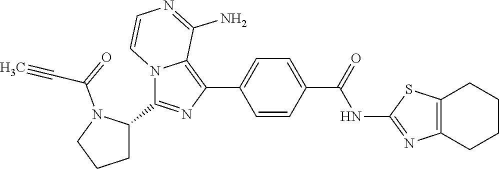 [SMILES: C#CC(=O)N1CCC[C@H]1c1nc(-c2ccc(C(=O)Nc3nc4c(s3)CCCC4)cc2)c2c(N)nccn12] |
| 35. | (S)-4-(3-(1-acryloylpyrrolidin-2-yl)-8- aminoimidazo[1,5-a]pyrazin-1-yl)-2- fluoro-N-(pyridin-2-yl)benzamide | 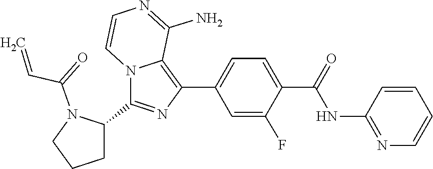 [SMILES: C=CC(=O)N1CCC[C@H]1c1nc(-c2ccc(C(=O)Nc3ccccn3)c(F)c2)c2c(N)nccn12] |
| 36. | (S)-4-(3-(1-Acryloylpyrrolidin-2-yl)-8- aminoimidazo[1,5-a]pyrazin-1-yl)-2- methoxy-N-(pyridin-2-yl)benzamide | 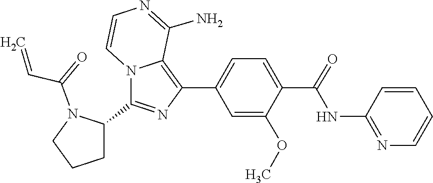 [SMILES: C=CC(=O)N1CCC[C@H]1c1nc(-c2ccc(C(=O)Nc3ccccn3)c(OC)c2)c2c(N)nccn12] |
| 37. | (S,E)-4-(8-Amino-3-(1-(4- (dimethylamino)but-2-enoyl)pyrrolidin- 2-yl)imidazo[1,5-a]pyrazin-1-yl)-N- (thiazol-2-yl)benzamide | 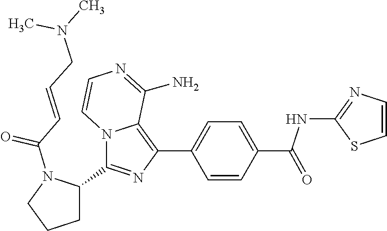 [SMILES: CN(C)C/C=C/C(=O)N1CCC[C@H]1c1nc(-c2ccc(C(=O)Nc3nccs3)cc2)c2c(N)nccn12] |
| 38. | (S)-4-(3-(1-Acryloylpiperidin-2-yl)-8- aminoimidazo[1,5-a]pyrazin-1-yl)-N-(4- fluoropyridin-2-yl)benzamide | 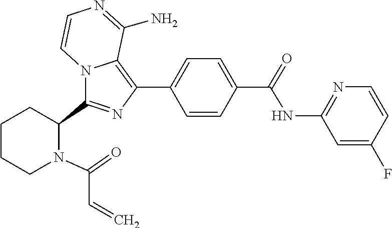 [SMILES: C=CC(=O)N1CCCC[C@H]1c1nc(-c2ccc(C(=O)Nc3cc(F)ccn3)cc2)c2c(N)nccn12] |
| 39. | (S)-4-(3-(1-Acryloylpiperidin-2-yl)-8- aminoimidazo[1,5-a]pyrazin-1-yl)-N-(4- cyanopyridin-2-yl)benzamide | 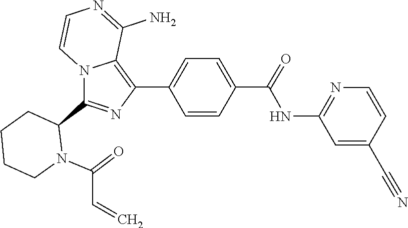 [SMILES: C=CC(=O)N1CCCC[C@H]1c1nc(-c2ccc(C(=O)Nc3cc(C#N)ccn3)cc2)c2c(N)nccn12] |
| 40. | (S)-4-(8-Amino-3-(1- (vinylsulfonyl)piperidin-2- yl)imidazo[1,5-a]pyrazin-1-yl)-N-(4- (trifluoromethyl)pyridin-2- yl)benzamide | 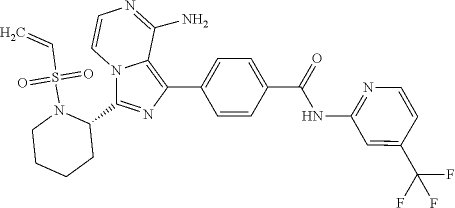 [SMILES: C=CS(=O)(=O)N1CCCC[C@H]1c1nc(-c2ccc(C(=O)Nc3cc(C(F)(F)F)ccn3)cc2)c2c(N)nccn12] |
| 41. | (S)-4-(3-(1-Acryloylpiperidin-2-yl)-8- aminoimidazo[1,5-a]pyrazin-1-yl)-N- (pyrimidin-2-yl)benzamide | 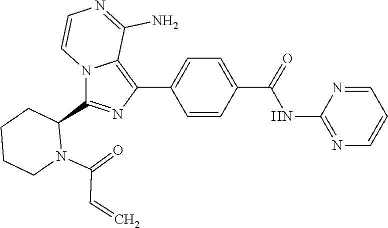 [SMILES: C=CC(=O)N1CCCC[C@H]1c1nc(-c2ccc(C(=O)Nc3ncccn3)cc2)c2c(N)nccn12] |
| 42. | (S)-4-(3-(1-Acryloylpiperidin-2-yl)-8- aminoimidazo[1,5-a]pyrazin-1-yl)-N-(4- methylpyrimidin-2-yl)benzamide | 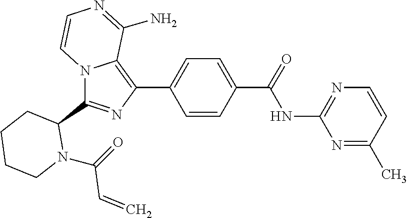 [SMILES: C=CC(=O)N1CCCC[C@H]1c1nc(-c2ccc(C(=O)Nc3nccc(C)n3)cc2)c2c(N)nccn12] |
| 43. | (S)-4-(8-Amino-3-(1-but-2- ynoylpiperidin-2-yl)imidazo[1,5-a] pyrazin-1-yl)-N-(pyrimidin-4- yl)benzamide | 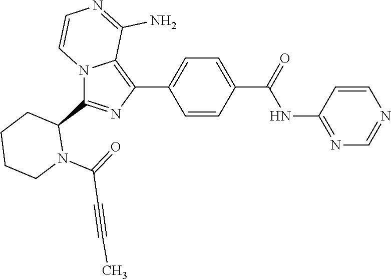 [SMILES: CC#CC(=O)N1CCCC[C@H]1c1nc(-c2ccc(C(=O)Nc3ccncn3)cc2)c2c(N)nccn12] |
| 44. | (S)-4-(8-Amino-3-(1-but-2- ynoylpiperidin-2-yl)imidazo[1,5-a] pyrazin-1-yl)-N-(pyridazin-3- yl)benzamide | 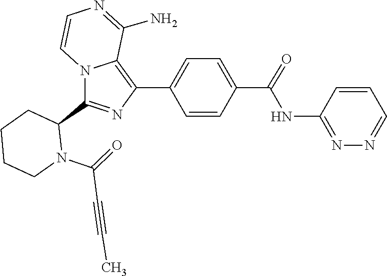 [SMILES: CC#CC(=O)N1CCCC[C@H]1c1nc(-c2ccc(C(=O)Nc3cccnn3)cc2)c2c(N)nccn12] |
| 45. | (S,E)-4-(8-Amino-3-(1-(4-methoxybut-2- enoyl)piperidin-2-yl)imidazo[1,5-a] pyrazin-1-yl)-N-(5-ethylthiazol-2- yl)benzamide | 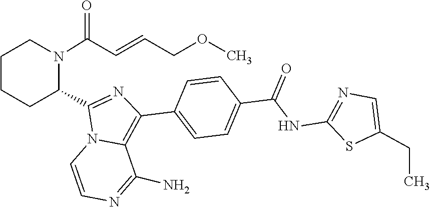 [SMILES: CCc1cnc(NC(=O)c2ccc(-c3nc([C@@H]4CCCCN4C(=O)/C=C/COC)n4ccnc(N)c34)cc2)s1] |
| 46. | (S)-4-(3-(1-Acryloylpiperidin-2-yl)-8- aminoimidazo[1,5-a]pyrazin-1-yl)-2- fluoro-N-(4-propylpyridin-2- yl)benzamide | 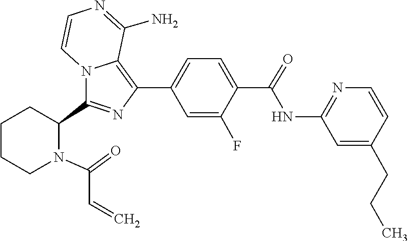 [SMILES: C=CC(=O)N1CCCC[C@H]1c1nc(-c2ccc(C(=O)Nc3cc(CCC)ccn3)c(F)c2)c2c(N)nccn12] |
| 47. | (S,E)-4-(8-Amino-3-(1-(4- (dimethylamino)but-2-enoyl)piperidin- 2-yl)imidazo[1,5-a]pyrazin-1-yl)-2- methoxy-N-(4-propylpyridin-2- yl)benzamide | 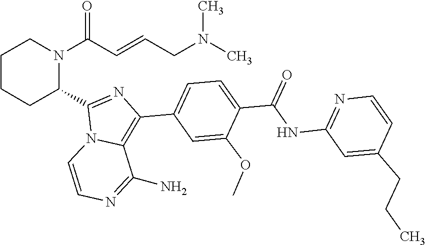 [SMILES: CCCc1ccnc(NC(=O)c2ccc(-c3nc([C@@H]4CCCCN4C(=O)/C=C/CN(C)C)n4ccnc(N)c34)cc2OC)c1] |
| 48. | 4-(8-Amino-3-((S)-1-but-2- ynoylpiperidin-2-yl)imidazo[1,5-a] pyrazin-1-yl)-3-methyl-N-(pyridin-2- yl)benzamide | 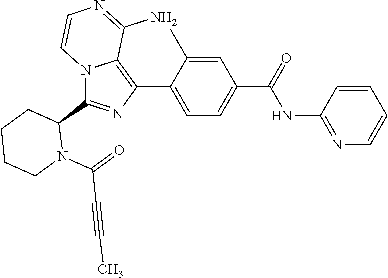 [SMILES: CC#CC(=O)N1CCCC[C@H]1c1nc(-c2ccc(C(=O)Nc3ccccn3)cc2C)c2c(N)nccn12] |
| 49. | 4-(3-(Acrylamidomethyl)-8- aminoimidazo[1,5-a]pyrazin-1-yl)-N- (pyridin-2-yl)benzamide | 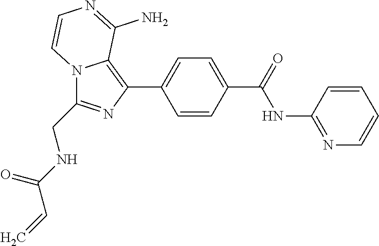 [SMILES: C=CC(=O)NCc1nc(-c2ccc(C(=O)Nc3ccccn3)cc2)c2c(N)nccn12] |
| 50. | (S)-4-(8-Amino-3-(1-but-2- ynamidoethyl)imidazo[1,5-a]pyrazin-1- yl)-N-(pyridin-2-yl)benzamide | 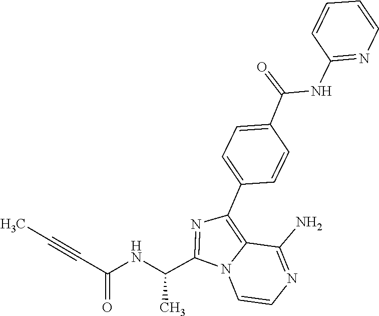 [SMILES: CC#CC(=O)N[C@@H](C)c1nc(-c2ccc(C(=O)Nc3ccccn3)cc2)c2c(N)nccn12] |
| 51. | (S)-S-(2-(2-(8-amino-1-(4-(pyridin-2- ylcarbamoyl)phenyl)imidazo[1,5- a]pyrazin-3-yl)pyrrolidin-1-yl)-2- oxoethyl)ethanethioate | 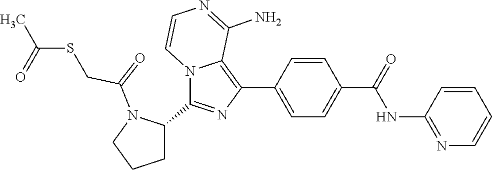 [SMILES: CC(=O)SCC(=O)N1CCC[C@H]1c1nc(-c2ccc(C(=O)Nc3ccccn3)cc2)c2c(N)nccn12] |
| 52. | (S)-4-(8-Amino-3-(1-(4-hydroxy-4- methylpent-2-ynoyl)pyrrolidin-2- yl)imidazo[1,5-a]pyrazin-1-yl)- N(pyridin-2-yl)benzamide | 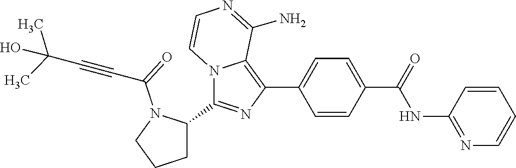 [SMILES: CC(C)(O)C#CC(=O)N1CCC[C@H]1c1nc(-c2ccc(C(=O)Nc3ccccn3)cc2)c2c(N)nccn12] |
| 53. | (S)-4-(8-Amino-3-(1-(6- chloropyrimidine-4-carbonyl)pyrrolidin- 2-yl)imidazo[1,5-a]pyrazin-1-yl)-N- (pyridin-2-yl)benzamide | 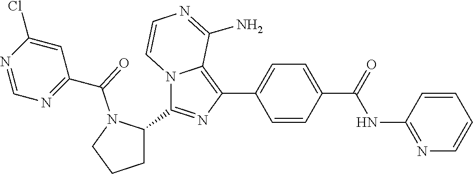 [SMILES: Nc1nccn2c([C@@H]3CCCN3C(=O)c3cc(Cl)ncn3)nc(-c3ccc(C(=O)Nc4ccccn4)cc3)c12] |
| 54. | (S)-4-(8-Amino-3-(1-pent-2- ynoylpyrrolidin-2-yl)imidazo[1,5-a] pyrazin-1-yl)-N-(pyridin-2-yl) benzamide | 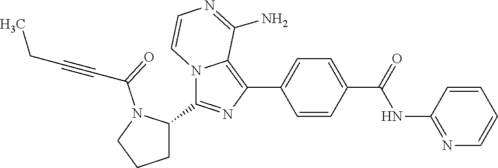 [SMILES: CCC#CC(=O)N1CCC[C@H]1c1nc(-c2ccc(C(=O)Nc3ccccn3)cc2)c2c(N)nccn12] |
| 55. | (S)-4-(8-Amino-3-(1-(3- cyclopropylpropioloyl)pyrrolidin-2- yl)imidazo[1,5-a]pyrazin-1-yl)-N- (pyridin-2-yl)benzamide | 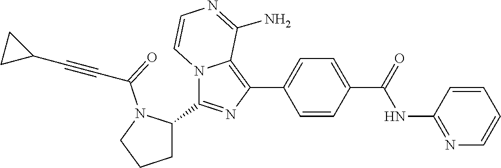 [SMILES: Nc1nccn2c([C@@H]3CCCN3C(=O)C#CC3CC3)nc(-c3ccc(C(=O)Nc4ccccn4)cc3)c12] |
| 56. | (S)-4-(8-Amino-3-(1-hex-2- ynoylpyrrolidin-2-yl)imidazo[1,5-a] pyrazin-1-yl)-N-(pyridin-2-yl)benzamide | 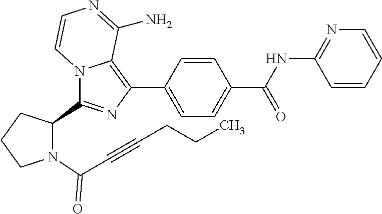 [SMILES: CCCC#CC(=O)N1CCC[C@H]1c1nc(-c2ccc(C(=O)Nc3ccccn3)cc2)c2c(N)nccn12] |
| 57. | 4-(3-(1-Acryloylazepan-2-yl)-8- aminoimidazo[1,5-a]pyrazin-1-yl)-N- (pyridin-2-yl)benzamide | 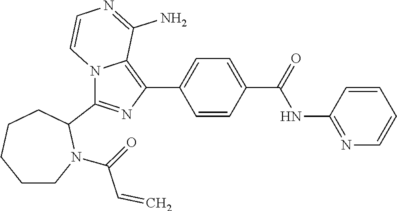 [SMILES: C=CC(=O)N1CCCCCC1c1nc(-c2ccc(C(=O)Nc3ccccn3)cc2)c2c(N)nccn12] |
| 58. | (R)-4-(8-Amino-3-(4-but-2- ynoylmorpholin-3-yl)imidazo[1,5-a] pyrazin-1-yl)-N-(pyridin-2-yl) benzamide | 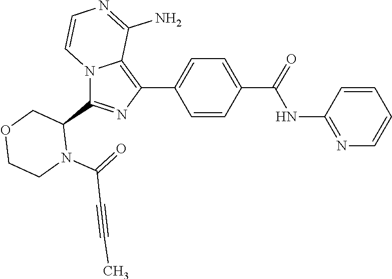 [SMILES: CC#CC(=O)N1CCOC[C@H]1c1nc(-c2ccc(C(=O)Nc3ccccn3)cc2)c2c(N)nccn12] |
| 59. | (S)-4-(8-amino-3-(1-(N-methylbut-2- ynamido)ethyl)imidazo[1,5-a]pyrazin-1- yl)-N-(4-(trifluoromethyl)pyridin-2- yl)benzamide | 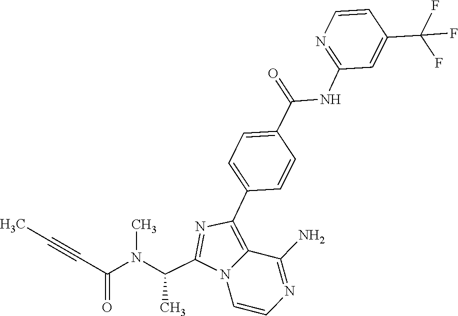 [SMILES: CC#CC(=O)N(C)[C@@H](C)c1nc(-c2ccc(C(=O)Nc3cc(C(F)(F)F)ccn3)cc2)c2c(N)nccn12] |
| 60. | (S)-4-(3-(1-acryloylpyrrolidin-2-yl)-8- aminoimidazo[1,5-a]pyrazin-1-yl)-N-(4- fluoropyridin-2-yl)benzamide | 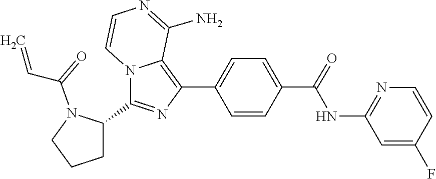 [SMILES: C=CC(=O)N1CCC[C@H]1c1nc(-c2ccc(C(=O)Nc3cc(F)ccn3)cc2)c2c(N)nccn12] |
| 61. | (S)-4-(3-(1-acryloylpyrrolidin-2-yl)-8- aminoimidazo[1,5-a]pyrazin-1-yl)-N-(4- (pyrrolidin-1-yl)pyridin-2-yl)benzamide | 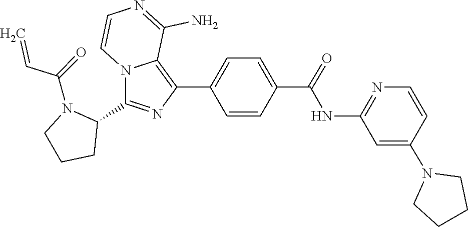 [SMILES: C=CC(=O)N1CCC[C@H]1c1nc(-c2ccc(C(=O)Nc3cc(N4CCCC4)ccn3)cc2)c2c(N)nccn12] |
| 62. | (S)-4-(8-amino-3-(1-but-2- ynoylpiperidin-2-yl)imidazo[1,5- a]pyrazin-1-yl)-N-(4-fluoropyridin-2- yl)benzamide | 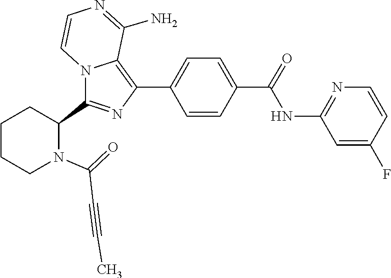 [SMILES: CC#CC(=O)N1CCCC[C@H]1c1nc(-c2ccc(C(=O)Nc3cc(F)ccn3)cc2)c2c(N)nccn12] |
| 63. | (S)-4-(8-amino-3-(1-but-2- ynoylpiperidin-2-yl)imidazo[1,5- a]pyrazin-1-yl)-N-(pyridine-2- yl)benzamide | 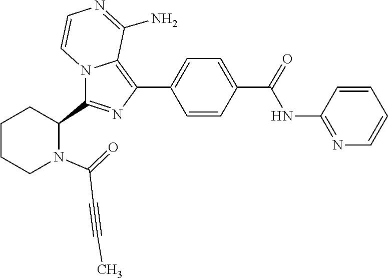 [SMILES: CC#CC(=O)N1CCCC[C@H]1c1nc(-c2ccc(C(=O)Nc3ccccn3)cc2)c2c(N)nccn12] |
| 64. | (S)-4-(3-(1-acrylpiperidin-2-yl)-8- aminoimidazo[1,5-a]pyrazin-1-yl)-N- (pyridine-2-yl)benzamide | 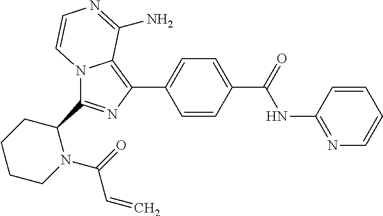 [SMILES: C=CC(=O)N1CCCC[C@H]1c1nc(-c2ccc(C(=O)Nc3ccccn3)cc2)c2c(N)nccn12] |
| 65. | (S)-4-(8-amino-3-(1-but-2- ynoylpyrrolidin-2-yl)imidazo[1,5- a]pyrazin-1-yl)-N-(4-propylpyridin-2- yl)benzamide | 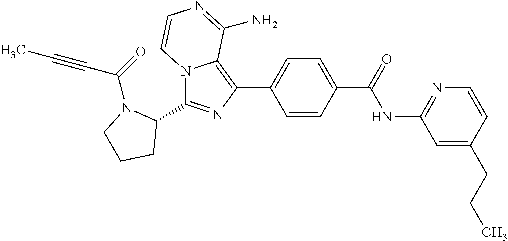 [SMILES: CC#CC(=O)N1CCC[C@H]1c1nc(-c2ccc(C(=O)Nc3cc(CCC)ccn3)cc2)c2c(N)nccn12] |
| 66. | (S,E)-4-(8-amino-3-(1-(4-methoxy-N- methylbut-2- enamido)ethyl)imidazo[1,5-a]pyrazin-1- yl)-N-(4-propylpyridin-2-yl)benzamide | 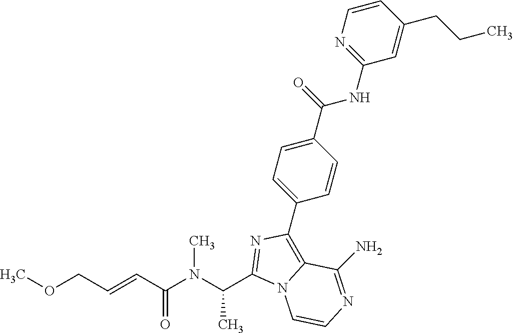 [SMILES: CCCc1ccnc(NC(=O)c2ccc(-c3nc([C@H](C)N(C)C(=O)/C=C/COC)n4ccnc(N)c34)cc2)c1] |
| 67. | (S)-4-(8-amino-3-(1- (vinylsulfonyl)piperidin-2- yl)imidazo[1,5-a]pyrazin-1-yl)-N-(4- propylpyridin-2-yl)benzamide | 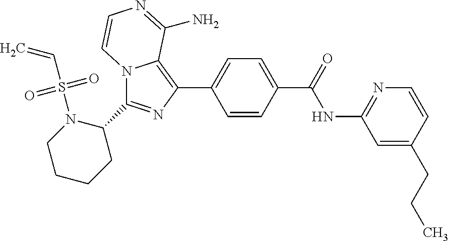 [SMILES: C=CS(=O)(=O)N1CCCC[C@H]1c1nc(-c2ccc(C(=O)Nc3cc(CCC)ccn3)cc2)c2c(N)nccn12] |
| 68. | (S)-4-(3-(1-acryloylpiperidin-2-yl)-8- aminoimidazo[1,5-a]pyrazin-1-yl)-N-(4- propylpyridin-2-yl)benzamide | 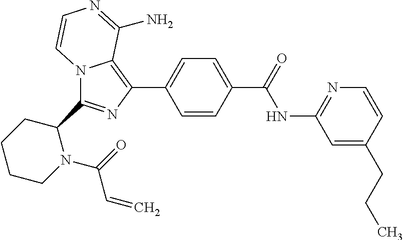 [SMILES: C=CC(=O)N1CCCC[C@H]1c1nc(-c2ccc(C(=O)Nc3cc(CCC)ccn3)cc2)c2c(N)nccn12] |
| 69. | (S)-4-(3-(1-acryloylpyrrolidin-2-yl)-8- aminoimidazo[1,5-a]pyrazin-1-yl)-N-(4- (trifluoromethyl)pyridin-2-yl)benzamide | 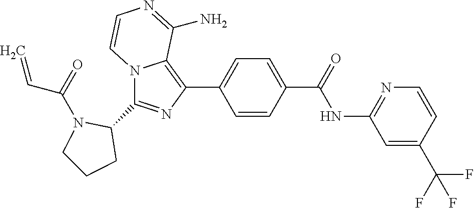 [SMILES: C=CC(=O)N1CCC[C@H]1c1nc(-c2ccc(C(=O)Nc3cc(C(F)(F)F)ccn3)cc2)c2c(N)nccn12] |
| 70. | (S)-4-(8-amino-3-(1-but-2- ynoylpiperidin-2-yl)imidazo[1,5- a]pyrazin-1-yl)-N-(4- (trifluoromethyl)pyridin-2- yl)benzamide | 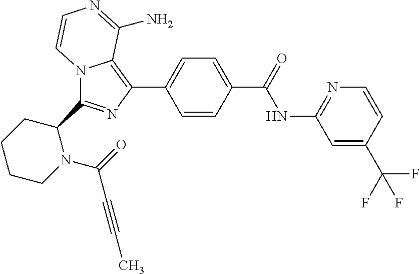 [SMILES: CC#CC(=O)N1CCCC[C@H]1c1nc(-c2ccc(C(=O)Nc3cc(C(F)(F)F)ccn3)cc2)c2c(N)nccn12] |
| 71. | (S)-4-(8-amino-3-(1-but-2- ynoylpiperidin-2-yl)imidazo[1,5- a]pyrazin-1-yl)-N-(4-propylpyridin-2- yl)benzamide | 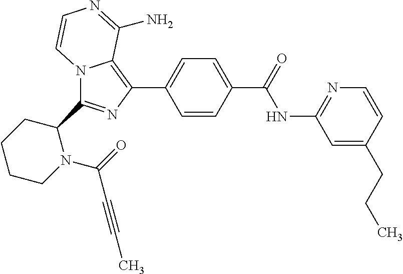 [SMILES: CC#CC(=O)N1CCCC[C@H]1c1nc(-c2ccc(C(=O)Nc3cc(CCC)ccn3)cc2)c2c(N)nccn12] |
| 72. | (S,E)-4-(8-amino-3-(1-(4- (dimethylamino)but-2-enoyl)pyrrolidin- 2-yl)imidazo[1,5-a]pyrazin-1-yl)-N-(4- isopropylpyridin-2-yl)benzamide | 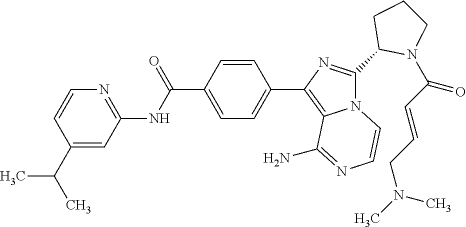 [SMILES: CC(C)c1ccnc(NC(=O)c2ccc(-c3nc([C@@H]4CCCN4C(=O)/C=C/CN(C)C)n4ccnc(N)c34)cc2)c1] |
| 73. | 4-(8-amino-3-((S)-1- (vinylsulfonyl)piperidin-2- yl)imidazo[1,5-a]pyrazin-1-yl)-3- methyl-N-(pyridin-2-yl) benzamide | 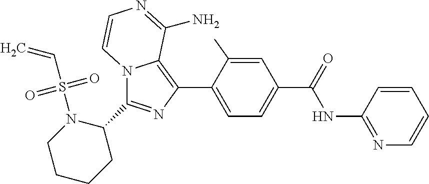 [SMILES: C=CS(=O)(=O)N1CCCC[C@H]1c1nc(-c2ccc(C(=O)Nc3ccccn3)cc2C)c2c(N)nccn12] |
| 74. | (S)-4-(8-amino-3-(1-but-2- ynoylpiperidin-2-yl)imidazo[1,5- a]pyrazin-1-yl)-2-fluoro-N-(4- propylpyridin-2-yl)benzamide | 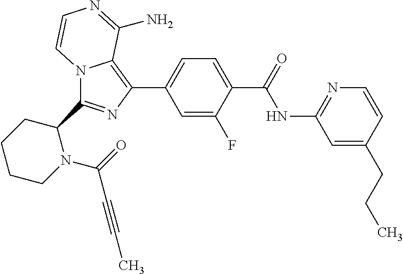 [SMILES: CC#CC(=O)N1CCCC[C@H]1c1nc(-c2ccc(C(=O)Nc3cc(CCC)ccn3)c(F)c2)c2c(N)nccn12] |
| 75. | (S,E)-4-(8-amino-3-(1-(4-methoxy-N- methylbut-2- enamido)ethyl)imidazo[1,5-a]pyrazin-1- yl)-N-(4-(trifluoromethyl)pyridin-2- yl)benzamide | 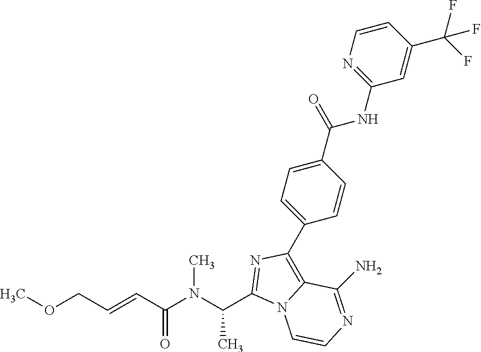 [SMILES: COC/C=C/C(=O)N(C)[C@@H](C)c1nc(-c2ccc(C(=O)Nc3cc(C(F)(F)F)ccn3)cc2)c2c(N)nccn12] |
| 76. | (S,E)-4-(8-amino-3-(1-(4- (dimethylamino)-N-methylbut-2- enamido)ethyl)imidazo[1,5-a] pyrazin-1-yl)-N-(4- propylpyridin-2-yl)benzamide | 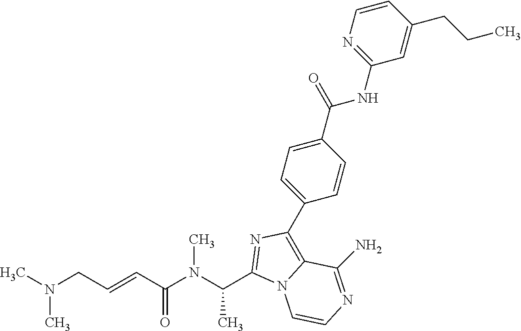 [SMILES: CCCc1ccnc(NC(=O)c2ccc(-c3nc([C@H](C)N(C)C(=O)/C=C/CN(C)C)n4ccnc(N)c34)cc2)c1] |
| 77. | (S,E)-4-(8-amino-3-(1-(4-(pyrrolidin-1- yl)but-2-enoyl)pyrrolidin-2- yl)imidazo[1,5-a]pyrazin-1-yl)-N-(4- propylpyridin-2-yl)benzamide | 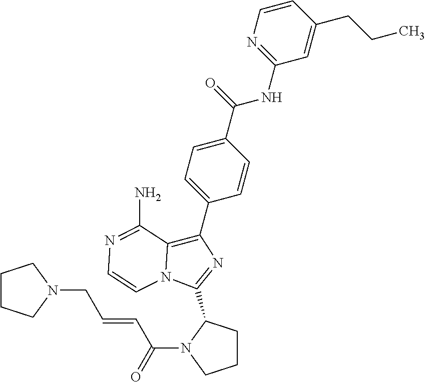 [SMILES: CCCc1ccnc(NC(=O)c2ccc(-c3nc([C@@H]4CCCN4C(=O)/C=C/CN4CCCC4)n4ccnc(N)c34)cc2)c1] |
| 78. | (S,E)-4-(8-amino-3-(1-(4- (dimethylamino)but-2-enoyl)piperidin- 2-yl)imidazo[1,5-a]pyrazin-1-yl)-N- (pyridin-2-yl)benzamide | 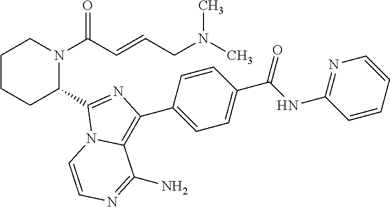 [SMILES: CN(C)C/C=C/C(=O)N1CCCC[C@H]1c1nc(-c2ccc(C(=O)Nc3ccccn3)cc2)c2c(N)nccn12] |
| 79. | (S)-4-(8-amino-3-(1-(2- chloropyrimidine-4-carbonyl)pyrrolidin- 2-yl)imidazo[1,5-a]pyrazin-1-yl)-N-(4- propylpyridin-2-yl)benzamide | 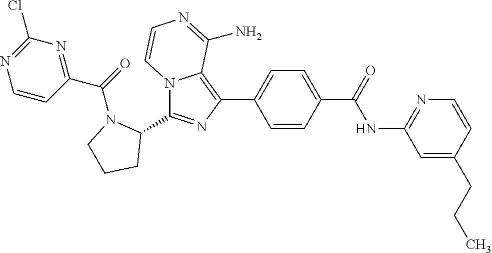 [SMILES: CCCc1ccnc(NC(=O)c2ccc(-c3nc([C@@H]4CCCN4C(=O)c4ccnc(Cl)n4)n4ccnc(N)c34)cc2)c1] |
| 80. | (S)-4-(3-(1-acrylamidoethyl)-8- aminoimidazo[1,5-a]pyrazin-1-yl)-N- (pyridin-2-yl)benzamide | 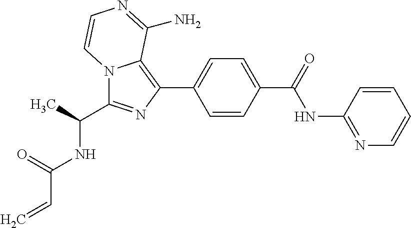 [SMILES: C=CC(=O)N[C@@H](C)c1nc(-c2ccc(C(=O)Nc3ccccn3)cc2)c2c(N)nccn12] |
| 81. | (S)-4-(3-(1-acryloylpyrrolidin-2-yl)-8- aminoimidazo[1,5-a]pyrazin-1-yl)-N- (thiazol-2-yl)benzamide | 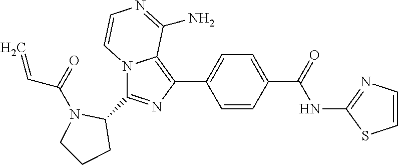 [SMILES: C=CC(=O)N1CCC[C@H]1c1nc(-c2ccc(C(=O)Nc3nccs3)cc2)c2c(N)nccn12] |
| 82. | (S)-4-(8-amino-3-(1-but-2- ynoylpyrrolidin-2-yl)imidazo[1,5- a]pyrazin-1-yl)-N-(4-isopropylpyridin- 2-yl)benzamide | 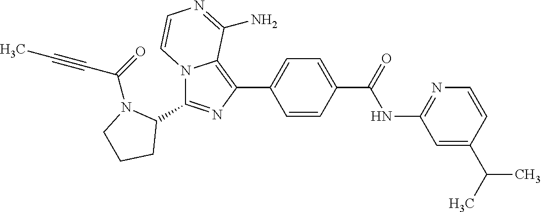 |
| 83. | (S)-4-(8-amino-3-(1-(2- chloropyrimidine-4-carbonyl) piperidin-2-yl)imidazo[1,5-a]pyrazin-1- yl)-N-(4-propylpyridin-2-yl) benzamide | 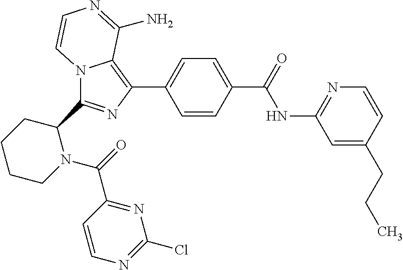 [SMILES: CCCc1ccnc(NC(=O)c2ccc(-c3nc([C@@H]4CCCCN4C(=O)c4ccnc(Cl)n4)n4ccnc(N)c34)cc2)c1] |
| 84. | (S,E)-4-(8-amino-3-(1-(4-methoxybut-2- enoyl)piperidin-2-yl)imidazo[1,5- a]pyrazin-1-yl)-N-(4- (trifluoromethyl)pyridin-2-yl) benzamide | 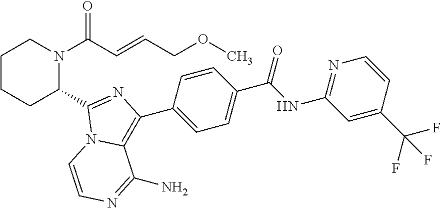 [SMILES: COC/C=C/C(=O)N1CCCC[C@H]1c1nc(-c2ccc(C(=O)Nc3cc(C(F)(F)F)ccn3)cc2)c2c(N)nccn12] |
| 85. | (S)-4-(3-(1-acryloylpiperidin-2-yl)-8- aminoimidazo[1,5-a]pyrazin-1-yl)-N-(4- (trifluoromethyl)pyridin-2-yl) benzamide | 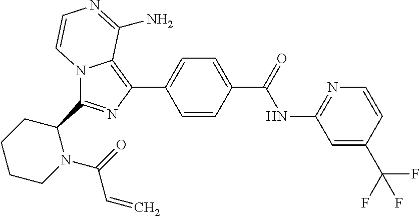 [SMILES: C=CC(=O)N1CCCC[C@H]1c1nc(-c2ccc(C(=O)Nc3cc(C(F)(F)F)ccn3)cc2)c2c(N)nccn12] |
| 86. | (S)-4-(8-amino-3-(1-but-2- ynoylpiperidin-2-yl)imidazo[1,5- a]pyrazin-1-yl)-2-methoxy-N-(4- propylpyridin-2-yl)benzamide | 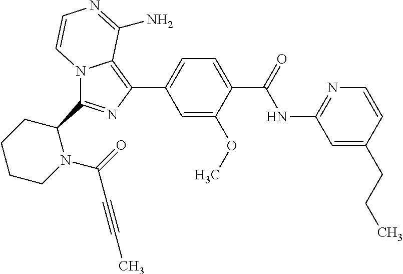 [SMILES: CC#CC(=O)N1CCCC[C@H]1c1nc(-c2ccc(C(=O)Nc3cc(CCC)ccn3)c(OC)c2)c2c(N)nccn12] |
| 87. | (S,E)-4-(8-amino-3-(1-(4-methoxybut- 2-enoyl)piperidin-2-yl)imidazo[1,5- a]pyrazin-1-yl)-2-methoxy-N-(4- propylpyridin-2-yl)benzamidee | 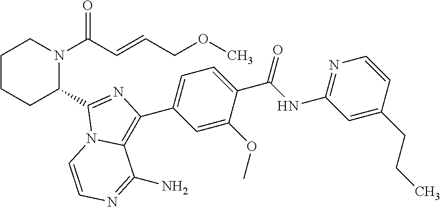 [SMILES: CCCc1ccnc(NC(=O)c2ccc(-c3nc([C@@H]4CCCCN4C(=O)/C=C/COC)n4ccnc(N)c34)cc2OC)c1] |
| 88. | (S)-4-(8-amino-3-(1-(2- chloropyrimidine-4-cabonyl)piperidin- 2-yl)imidazo[1,5-a]pyrazin-1-yl)-N-(4- (trifluoromethyl)pyridin-2- yl)benzamide | 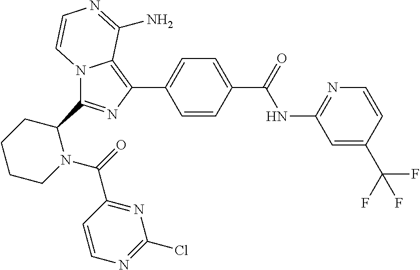 [SMILES: Nc1nccn2c([C@@H]3CCCCN3C(=O)c3ccnc(Cl)n3)nc(-c3ccc(C(=O)Nc4cc(C(F)(F)F)ccn4)cc3)c12] |
| 89. | (S)-4-(8-amino-3-(1-but-2- ynoylpiperidin-2-yl)imidazo[1,5- a]pyrazin-1-yl)-N-(5-ethylthiazol-2- yl)benzamide | 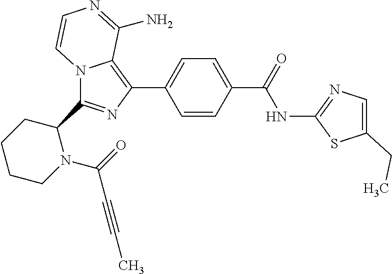 [SMILES: CC#CC(=O)N1CCCC[C@H]1c1nc(-c2ccc(C(=O)Nc3ncc(CC)s3)cc2)c2c(N)nccn12] |
| 90. | (S)-4-(3-(1-acryloylpiperidin-2-yl)-8- aminoimidazo[1,5-a]pyrazin-1-yl)-N-(5- ethylthiazol-2-yl)benzamide | 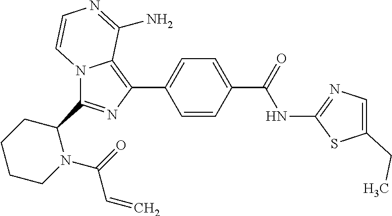 [SMILES: C=CC(=O)N1CCCC[C@H]1c1nc(-c2ccc(C(=O)Nc3ncc(CC)s3)cc2)c2c(N)nccn12] |
| 91. | (S)-4-(8-amino-3-(1-(2- chloropyrimidine-4-carbonyl)piperidin- 2-yl)imidazo[1,5-a]pyrazin-1-yl)-N-(5- ethylthiazol-2-yl)benzamide | 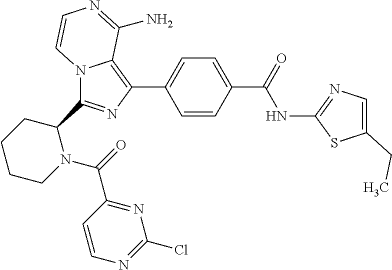 [SMILES: CCc1cnc(NC(=O)c2ccc(-c3nc([C@@H]4CCCCN4C(=O)c4ccnc(Cl)n4)n4ccnc(N)c34)cc2)s1] |
| 92. | (R,E)-4-(8-amino-3-(4-(4-methoxybut-2- enoyl)morpholin-3-yl)imidazo[1,5- a]pyrazin-1-yl)-N-(pyridin-2- yl)benzamide | 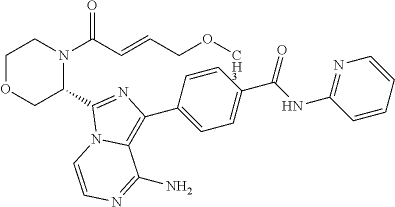 [SMILES: COC/C=C/C(=O)N1CCOC[C@H]1c1nc(-c2ccc(C(=O)Nc3ccccn3)cc2)c2c(N)nccn12] |
| 93. | (S,E)-4-(8-amino-3-(1-(4-methoxybut-2- enoyl)piperidin-2-yl)imidazo[1,5- a]pyrazin-1-yl)-N-(4-propylpyridin-2- yl)benzamide | 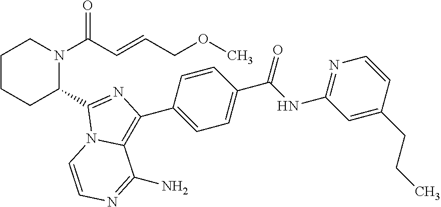 [SMILES: CCCc1ccnc(NC(=O)c2ccc(-c3nc([C@@H]4CCCCN4C(=O)/C=C/COC)n4ccnc(N)c34)cc2)c1] |
| 94. | (S)-4-(3-(1-acryloylpyrrolidin-2-yl)-8- aminoimidazo[1,5-a]pyrazin-1-yl)-N-(4- cyanopyridin-2-yl)benzamide | 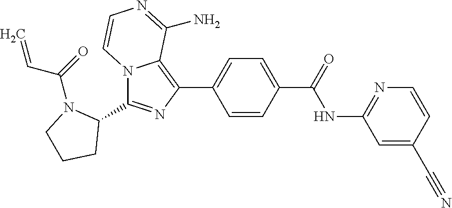 [SMILES: C=CC(=O)N1CCC[C@H]1c1nc(-c2ccc(C(=O)Nc3cc(C#N)ccn3)cc2)c2c(N)nccn12] |
| 95. | (S)-4-(8-amino-3-(1-but-2- ynoylpyrrolidin-2-yl)imidazo[1,5- a]pyrazin-1-yl)-N-(4-methoxypyridin- 2-yl)benzamide | 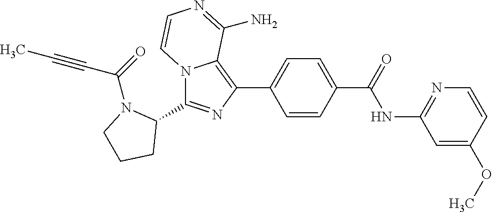 [SMILES: CC#CC(=O)N1CCC[C@H]1c1nc(-c2ccc(C(=O)Nc3cc(OC)ccn3)cc2)c2c(N)nccn12] |
| 96. | (S)-4-(3-(1-acryloylpyrrolidin-2-yl)-8- aminoimidazo[1,5-a]pyrazin-1-yl)-N-(4- methylpyridin-2-yl)benzamide | 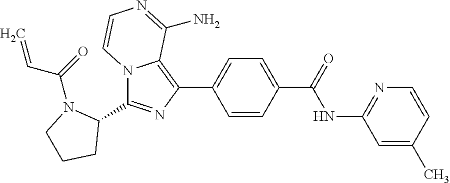 [SMILES: C=CC(=O)N1CCC[C@H]1c1nc(-c2ccc(C(=O)Nc3cc(C)ccn3)cc2)c2c(N)nccn12] |
| 97. | (S)-4-(3-(1-acryloylpyrrolidin-2-yl)-8- aminoimidazo[1,5-a]pyrazin-1-yl)-N-(4- propylpyridin-2-yl)benzamide | 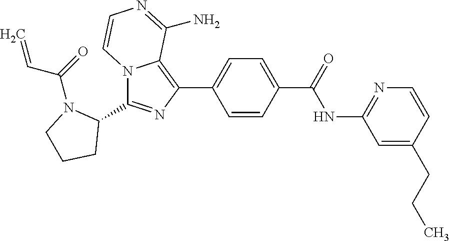 [SMILES: C=CC(=O)N1CCC[C@H]1c1nc(-c2ccc(C(=O)Nc3cc(CCC)ccn3)cc2)c2c(N)nccn12] |
| 98. | (S)-4-(3-(1-acryloylpyrrolidin-2-yl)-8- aminoimidazo[1,5-a]pyrazin-1-yl)-N-(4- ethylpyridin-2-yl)benzamide | 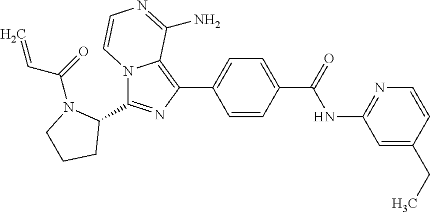 [SMILES: C=CC(=O)N1CCC[C@H]1c1nc(-c2ccc(C(=O)Nc3cc(CC)ccn3)cc2)c2c(N)nccn12] |
| 99. | (S,E)-4-(8-amino-3-(1-(4- (dimethylamino)but-2-enoyl)pyrrolidin- 2-yl)imidazo[1,5-a]pyrazin-1-yl)-N- (pyridin-2-yl)benzamide | 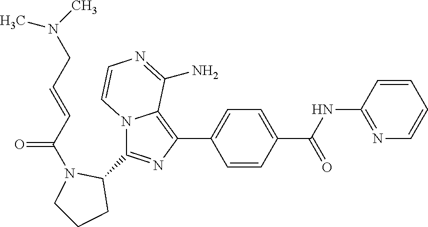 [SMILES: CN(C)C/C=C/C(=O)N1CCC[C@H]1c1nc(-c2ccc(C(=O)Nc3ccccn3)cc2)c2c(N)nccn12] |
| 100. | (S,E)-4-(8-amino-3-(1-(4-methoxybut-2- enoyl)pyrrolidin-2-yl)imidazo[1,5- a]pyrazin-1-yl)-N-(4- (trifluoromethyl)pyridin-2- yl)benzamide | 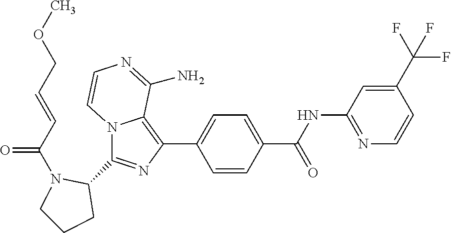 [SMILES: COC/C=C/C(=O)N1CCC[C@H]1c1nc(-c2ccc(C(=O)Nc3cc(C(F)(F)F)ccn3)cc2)c2c(N)nccn12] |
| 101. | (S)-4-(8-amino-3-(1-(2- chloropyrimidine-4-carbonyl)pyrrolidin- 2-yl)imidazo[1,5-a]pyrazin-1-yl)-N-(4- methylpyridin-2-yl)benzamide | 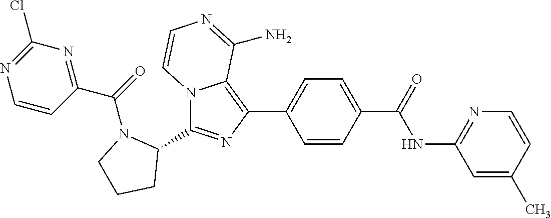 [SMILES: Cc1ccnc(NC(=O)c2ccc(-c3nc([C@@H]4CCCN4C(=O)c4ccnc(Cl)n4)n4ccnc(N)c34)cc2)c1] |
| 102. | (S)-4-(8-amino-3-(1-but-2- ynoylpyrrolidin-2-yl)imidazo[1,5-a] pyrazin-1-yl)-N-(4-cyanopyridin-2- yl)benzamide | 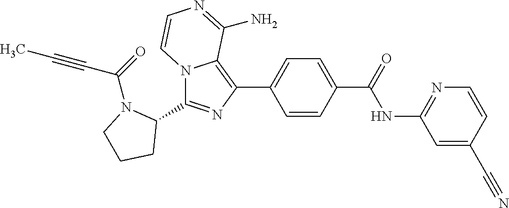 [SMILES: CC#CC(=O)N1CCC[C@H]1c1nc(-c2ccc(C(=O)Nc3cc(C#N)ccn3)cc2)c2c(N)nccn12] |
| 103. | (S)-4-(8-amino-3-(1-but-2- ynoylpyrrolidin-2-yl)imidazo[1,5- a]pyrazin-1-yl)-N-(4-ethylpyridin-2- yl)benzamide | 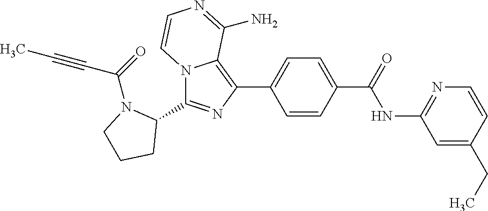 [SMILES: CC#CC(=O)N1CCC[C@H]1c1nc(-c2ccc(C(=O)Nc3cc(CC)ccn3)cc2)c2c(N)nccn12] |
| 104. | (S)-4-(8-amino-3-(1-but-2- ynoylpyrrolidin-2-yl)imidazo[1,5- a]pyrazin-1-yl)-N-(4-phenylpyridin-2- yl)benzamide | 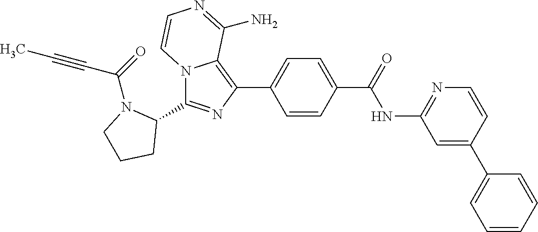 [SMILES: CC#CC(=O)N1CCC[C@H]1c1nc(-c2ccc(C(=O)Nc3cc(-c4ccccc4)ccn3)cc2)c2c(N)nccn12] |
| 105. | (S)-4-(3-(1-acryloylpyrrolidin-2-yl)-8- aminoimidazo[1,5-a]pyrazin-1-yl)N-(4- phenylpyridin-2-yl)benzamide | 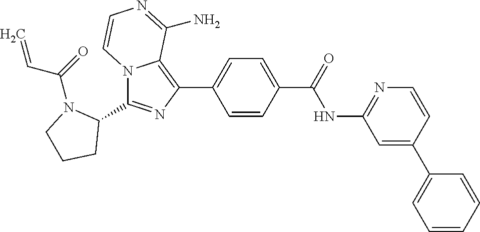 [SMILES: C=CC(=O)N1CCC[C@H]1c1nc(-c2ccc(C(=O)Nc3cc(-c4ccccc4)ccn3)cc2)c2c(N)nccn12] |
| 106. | (R,E)-1-(3-(4-amino-3-(4- phenoxyphenyl)-1H-pyrazolo[3,4- d]pyrimidin-1-yl)piperidin-1-yl)-4- (dimethylamino)but-2-en-1-one | 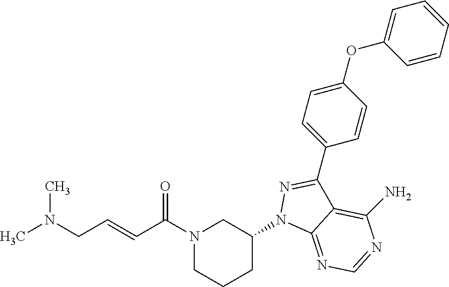 [SMILES: CN(C)C/C=C/C(=O)N1CCC[C@@H](n2nc(-c3ccc(Oc4ccccc4)cc3)c3c(N)ncnc32)C1] |
| 107. | (E)-1-(3-(4-amino-3-(4-phenoxyphenyl)- 1H-pyrazolo[3,4-d]pyrimidin-1- yl)piperidin-1-yl)-4-morpholinobut-2- en-1-one | 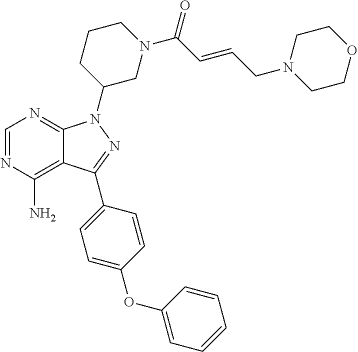 [SMILES: Nc1ncnc2c1c(-c1ccc(Oc3ccccc3)cc1)nn2C1CCCN(C(=O)/C=C/CN2CCOCC2)C1] |
| 108. | 1-(4-(4-amino-3-(4-phenoxyphenyl)-1H- pyrazolo[3,4-d]pyrimidin-1-yl)piperidin- 1-yl)prop-2-en-1-one | 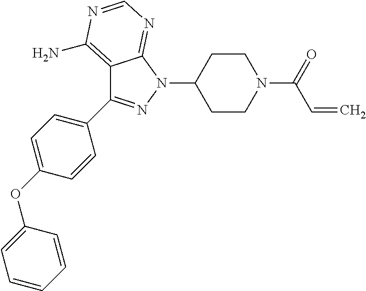 [SMILES: C=CC(=O)N1CCC(n2nc(-c3ccc(Oc4ccccc4)cc3)c3c(N)ncnc32)CC1] |
| 109. | (E)-1-(4-(4-amino-3-(4-phenoxyphenyl)- 1H-pyrazolo[3,4-d]pyrimidin-1- yl)piperidin-1-yl)-4-(dimethylamino) but-2-en-1-one | 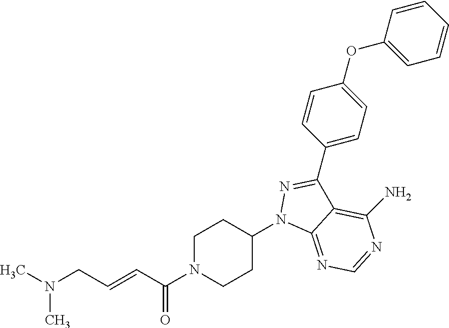 [SMILES: CN(C)C/C=C/C(=O)N1CCC(n2nc(-c3ccc(Oc4ccccc4)cc3)c3c(N)ncnc32)CC1] |
| 110. | (E)-N-((1s,4s)-4-(4-amino-3-(4- phenoxyphenyl)-1H-pyrazolo[3,4- d]pyrimidin-1-yl)cyclohexyl)-4- (dimethylamino)but-2-enamide | 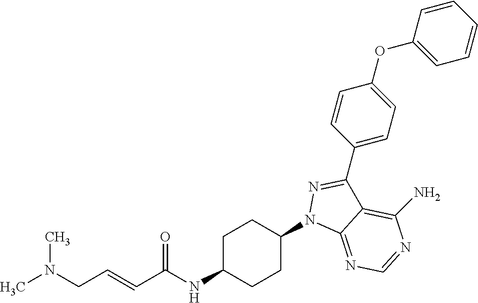 [SMILES: CN(C)C/C=C/C(=O)N[C@H]1CC[C@@H](n2nc(-c3ccc(Oc4ccccc4)cc3)c3c(N)ncnc32)CC1] |
| 111. | 1-(4-(4-amino-3-(4-phenoxyphenyl)-1H- pyrazolo[3,4-d]pyrimidin-1-yl)piperidin- 1-yl)prop-2-en-1-one | 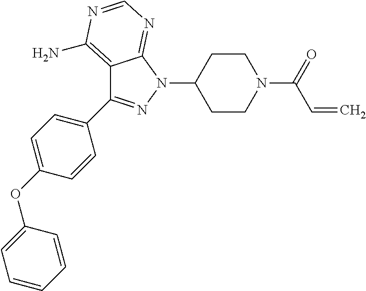 [SMILES: C=CC(=O)N1CCC(n2nc(-c3ccc(Oc4ccccc4)cc3)c3c(N)ncnc32)CC1] |
| 112. | N-((1r,4r)-4-(4-amino-3-(4- phenoxyphenyl)-1H-pyrazolo[3,4- d]pyrimidin-1-yl)cyclohexyl)acrylamide | 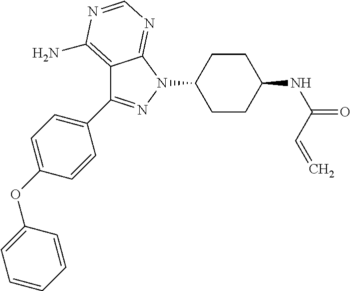 [SMILES: C=CC(=O)N[C@H]1CC[C@H](n2nc(-c3ccc(Oc4ccccc4)cc3)c3c(N)ncnc32)CC1] |
| 113. | (E)-1-((R)-2-((4-amino-3-(4- phenoxyphenyl)-1H-pyrazolo[3,4- d]pyrimidin-1-yl)methyl)pyrrolidin-1- yl)-4-(dimethylamino)but-2-en-1-one | 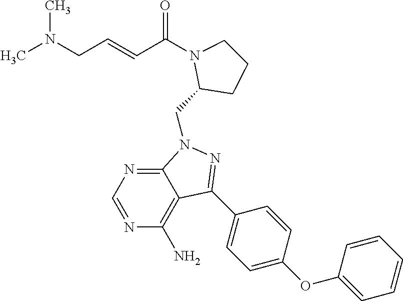 [SMILES: CN(C)C/C=C/C(=O)N1CCC[C@@H]1Cn1nc(-c2ccc(Oc3ccccc3)cc2)c2c(N)ncnc21] |
| 114. | (E)-1-((S)-2-((4-amino-3-(4- phenoxyphenyl)-1H-pyrazolo[3,4- d]pyrimidin-1-yl)methyl)pyrrolidin-1- yl)-4-(dimethylamino)but-2-en-1-one | 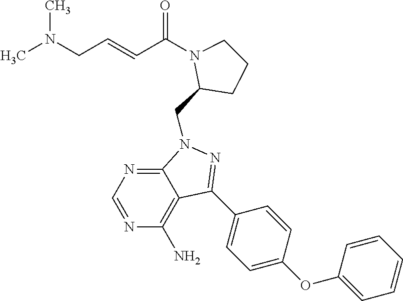 [SMILES: CN(C)C/C=C/C(=O)N1CCC[C@H]1Cn1nc(-c2ccc(Oc3ccccc3)cc2)c2c(N)ncnc21] |
| 115. | 1-((R)-2-((4-amino-3-(4- phenoxyphenyl)-1H-pyrazolo[3,4- d]pyrimidin-1-yl)methyl)pyrrolidin-1- yl)prop-2-en-1-one | 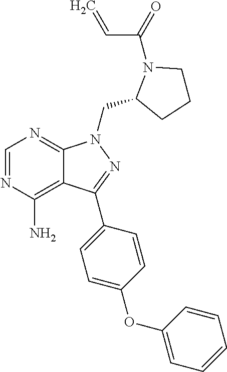 [SMILES: C=CC(=O)N1CCC[C@@H]1Cn1nc(-c2ccc(Oc3ccccc3)cc2)c2c(N)ncnc21] |
| 116. | 1-((S)-2-((4-amino-3-(4- phenoxyphenyl)-1H-pyrazolo[3,4- d]pyrimidin-1-yl)methyl)pyrrolidin-1- yl)prop-2-en-1-one | 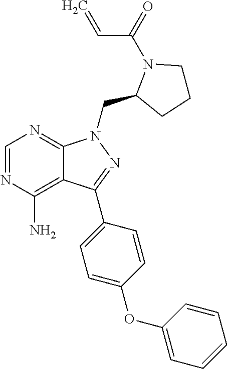 [SMILES: C=CC(=O)N1CCC[C@H]1Cn1nc(-c2ccc(Oc3ccccc3)cc2)c2c(N)ncnc21] |
| 117. | 1-((R)-2-((4-amino-3-(4- phenoxyphenyl)-1H-pyrazolo[3,4- d]pyrimidin-1-yl)methyl)pyrrolidin-1- yl)but-2-yn-1-one | 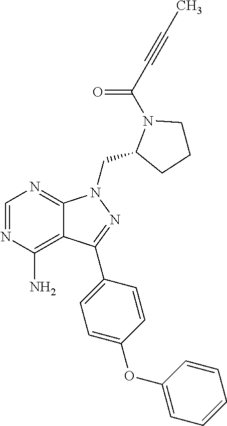 [SMILES: CC#CC(=O)N1CCC[C@@H]1Cn1nc(-c2ccc(Oc3ccccc3)cc2)c2c(N)ncnc21] |
| 118. | 1-((S)-2-((4-amino-3-(4-phenoxyphenyl)- 1H-pyrazolo[3,4-d]pyrimidin-1- yl)methyl)pyrrolidin-1-yl)but-2-yn-1- one | 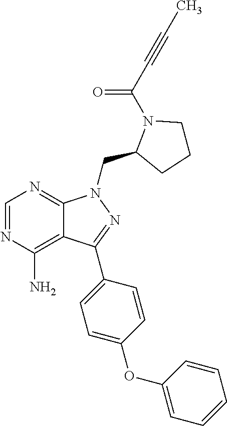 [SMILES: CC#CC(=O)N1CCC[C@H]1Cn1nc(-c2ccc(Oc3ccccc3)cc2)c2c(N)ncnc21] |
| 119. | 1-((R)-3-(4-amino-3-(4-phenoxyphenyl)- 1H-pyrazolo[3,4-d]pyrimidin-1- yl)piperidin-1-yl)but-2-yn-1-one | 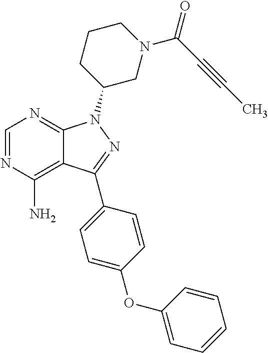 [SMILES: CC#CC(=O)N1CCC[C@@H](n2nc(-c3ccc(Oc4ccccc4)cc3)c3c(N)ncnc32)C1] |
| 120. | (E)-N-((1r,4r)-4-(4-amino-3-(4- phenoxyphenyl)-1H-pyrazolo[3,4- d]pyrimidin-1-yl)cyclohexyl-4- (dimethylamino)but-2-enamide | 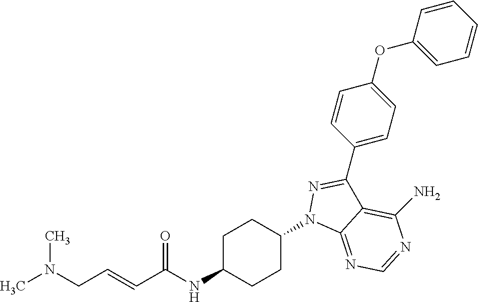 [SMILES: CN(C)C/C=C/C(=O)N[C@H]1CC[C@H](n2nc(-c3ccc(Oc4ccccc4)cc3)c3c(N)ncnc32)CC1] |
| 121. | N-(2-(4-amino-3-(4-phenoxyphenyl)-1H- pyrazolo[3,4-d]pyrimidin-1-yl)ethyl)-N- methylacrylamide | 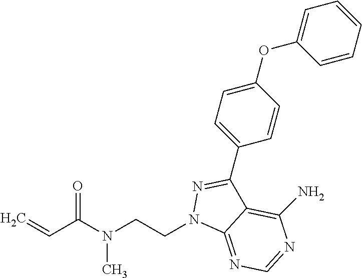 [SMILES: C=CC(=O)N(C)CCn1nc(-c2ccc(Oc3ccccc3)cc2)c2c(N)ncnc21] |
| 122. | (E)-1-(4-(4-amino-3-(4-phenoxyphenyl)- 1H-pyrazolo[3,4-d]pyrimidin-1-yl)-4- morpholino)but-2-en-1-one | 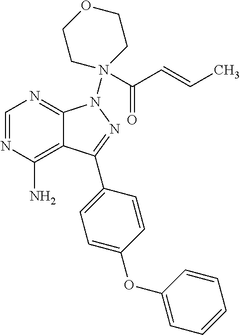 |
| 123. | (E)-1-(2-((S)-2-((4-amino-3-(4- phenoxyphenyl)-1H-pyrazolo[3,4- d]pyrimidin-1-yl)methyl)pyrrolidin-1- yl)morpholino)but-2-en-1-one | 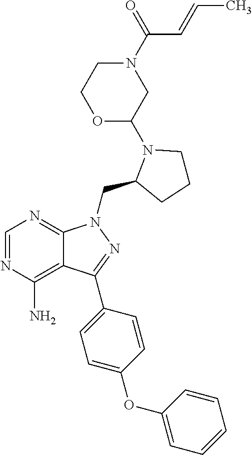 [SMILES: C/C=C/C(=O)N1CCOC(N2CCC[C@H]2Cn2nc(-c3ccc(Oc4ccccc4)cc3)c3c(N)ncnc32)C1] |
| 124. | N-((1s,4s)-4-(4-amino-3-(4- phenoxyphenyl)-1H-pyrazolo[3,4- d]pyrimidin-1-yl)cyclohexyl)but-2- ynamide | 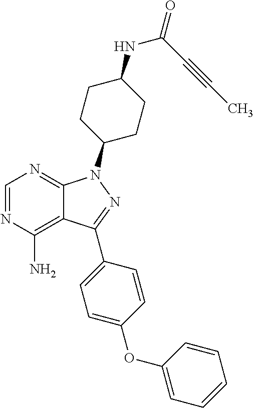 [SMILES: CC#CC(=O)N[C@H]1CC[C@@H](n2nc(-c3ccc(Oc4ccccc4)cc3)c3c(N)ncnc32)CC1] |
| 125. | N-(2-(4-amino-3-(4-phenoxyphenyl)-1H- pyrazolo[3,4-d]pyrimidin-1- yl)ethyl)acrylamide | 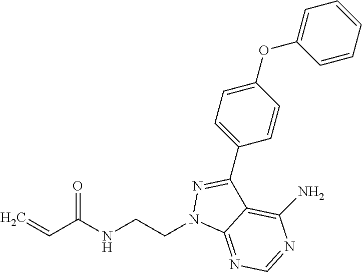 [SMILES: C=CC(=O)NCCn1nc(-c2ccc(Oc3ccccc3)cc2)c2c(N)ncnc21] |
| 126. | (E)-1-((R)-3-(4-amino-3-(4- phenoxyphenyl)-1H-pyrazolo[3,4- d]pyrimidin-1-yl)piperidin-1-yl)-4- morpholinobut-2-en-1-one | 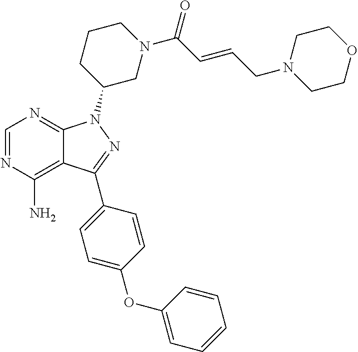 [SMILES: Nc1ncnc2c1c(-c1ccc(Oc3ccccc3)cc1)nn2[C@@H]1CCCN(C(=O)/C=C/CN2CCOCC2)C1] |
| 127. | (E)-N-((1s,4s)-4-(4-amino-3-(4- phenoxyphenyl)-1H-pyrazolo[3,4- d]pyrimidin-1-yl)cyclohexyl)-4- morpholinobut-2-enamide | 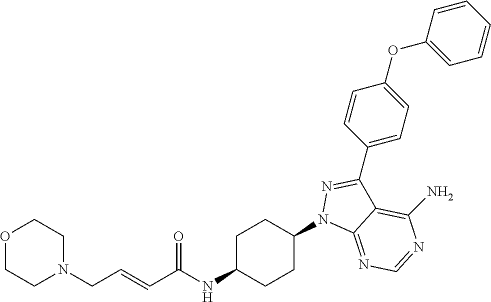 [SMILES: Nc1ncnc2c1c(-c1ccc(Oc3ccccc3)cc1)nn2[C@H]1CC[C@@H](NC(=O)/C=C/CN2CCOCC2)CC1] |
| 128. | 1-(4-(((6-amino-5-(4- phenoxyphenyl)pyrimidin-4- yl)amino)methyl)-4-fluoropiperidin-1- yl)prop-2-en-1-one | 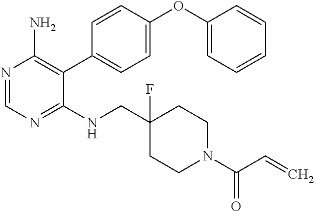 [SMILES: C=CC(=O)N1CCC(F)(CNc2ncnc(N)c2-c2ccc(Oc3ccccc3)cc2)CC1] |
| 129. | N-[3-[[5-fluoro-2-[4-(2- methoxyethoxy)anilino]pyrimidin-4- yl]amino]phenyl]propyl-2-enamide | 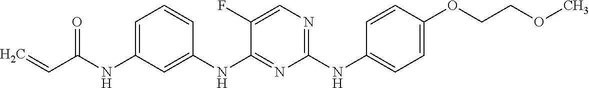 [SMILES: C=CC(=O)Nc1cccc(Nc2nc(Nc3ccc(OCCOC)cc3)ncc2F)c1] |
| 130. | 6-amino-9-[(3R)-1-but-2- ynoylpyrrolidin-3-yl]-7-(4- phenoxyphenyl)purin-8-one | 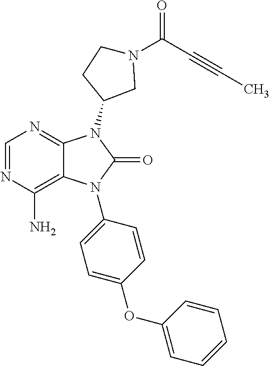 [SMILES: CC#CC(=O)N1CC[C@@H](n2c(=O)n(-c3ccc(Oc4ccccc4)cc3)c3c(N)ncnc32)C1] |
| 131. | (7S)-2-(4-phenoxyphenyl)-7-(1-prop-2- enoylpiperidin-4-yl)-4,5,6,7- tetrahydropyrazolo[1,5-a]pyrimidine-3- carboxamide | 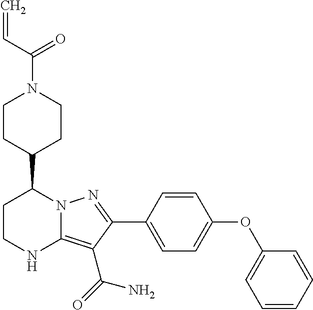 [SMILES: C=CC(=O)N1CCC([C@@H]2CCNc3c(C(N)=O)c(-c4ccc(Oc5ccccc5)cc4)nn32)CC1] |
| 132. | Orelabrutinib (2-(4-phenoxyphenyl)-6- (1-prop-2-enoylpiperidin-4-yl)pyridine- 3-carboxamid) | 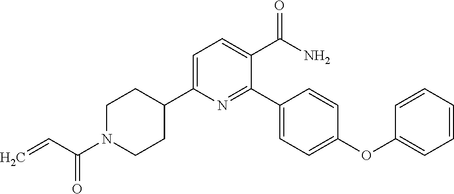 [SMILES: C=CC(=O)N1CCC(c2ccc(C(N)=O)c(-c3ccc(Oc4ccccc4)cc3)n2)CC1] |
| 133. | Remibrutinib (N-[3-[6-amino-5-[2- [methyl(prop-2- enoyl)amino]ethoxy]pyrimidin-4-yl]-5- fluoro-2-methylphenyl]-4-cyclopropyl- 2-fluorobenzamide | 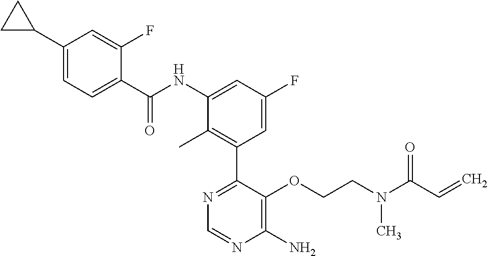 [SMILES: C=CC(=O)N(C)CCOc1c(N)ncnc1-c1cc(F)cc(NC(=O)c2ccc(C3CC3)cc2F)c1C] |
| 134. | Loxo-305 (1H-Pyrazole-4-carboxamide, 5-amino-3-[4-[[(5-fluoro-2- methoxybenzoyl)amino]methyl]phenyl]- 1-[(1S)-2,2,2-trifluoro-1-methylethyl]) | 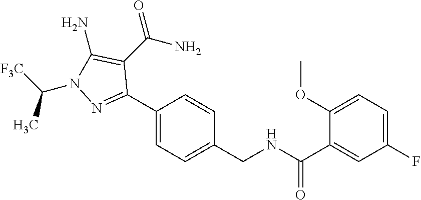 [SMILES: COc1ccc(F)cc1C(=O)NCc1ccc(-c2nn([C@@H](C)C(F)(F)F)c(N)c2C(N)=O)cc1] |
| 135. | TG-1701 (4-amino-1-[(3R)-1-but-2- ynoylpyrrolidin-3-yl]-3-[4-(2,6- difluorophenoxy)phenyl]-6H- pyrrolo[2,3-d]pyridazin-7-one) | 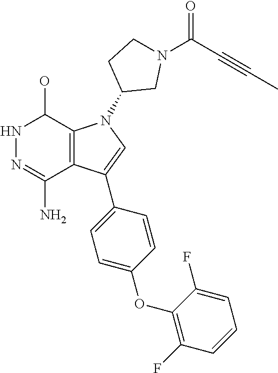 [SMILES: CC#CC(=O)N1CC[C@@H](n2cc(-c3ccc(Oc4c(F)cccc4F)cc3)c3c2C(O)NN=C3N)C1] |
| 136. | N-((1s,4s)-4-(4-amino-3-(4- phenoxyphenyl)-1H-pyrazolo[3,4- d]pyrimidin-1-yl)cyclohexyl)acrylamide | 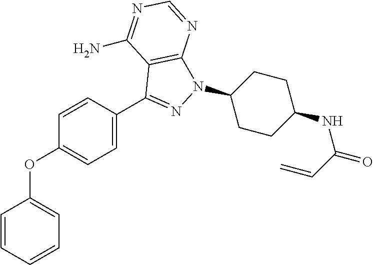 [SMILES: C=CC(=O)N[C@H]1CC[C@@H](n2nc(-c3ccc(Oc4ccccc4)cc3)c3c(N)ncnc32)CC1] |
| 137. | N-((1r,4r)-4-(4-amino-3-(4- phenoxyphenyl)-1H-pyrazolo[3,4- d]pyrimidin-1-yl)cyclohexyl)but-2- ynamide | 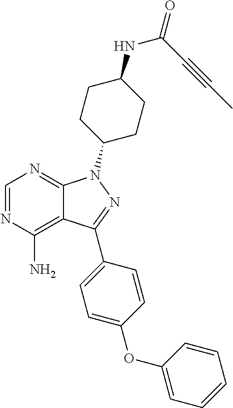 [SMILES: CC#CC(=O)N[C@H]1CC[C@H](n2nc(-c3ccc(Oc4ccccc4)cc3)c3c(N)ncnc32)CC1] |
| 138. | (E)-N-((1r,4r)-4-(4-amino-3-(4- phenoxyphenyl)-1H-pyrazolo[3,4- d]pyrimidin-1-yl)cyclohexyl)-4- morpholinobut-2-enamide | 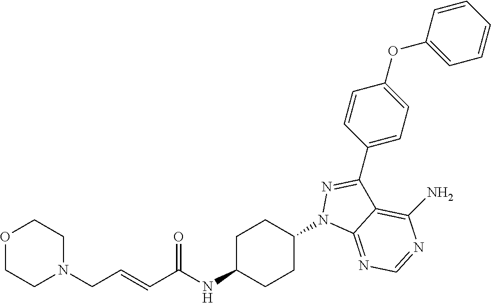 [SMILES: Nc1ncnc2c1c(-c1ccc(Oc3ccccc3)cc1)nn2[C@H]1CC[C@H](NC(=O)/C=C/CN2CCOCC2)CC1] |

In some embodiments, the BTK inhibitor is TG-1701 or Loxo-305.

In an embodiment, the BTK inhibitor is 1-(4-(((6-amino-5-(4-phenoxyphenyl)pyrimidin-4-yl)amino)methyl)-4-fluoropiperidin-1-yl)prop-2-en-1-one or a pharmaceutically acceptable salt thereof.

In an embodiment, the disclosure relates to a use of a composition comprising a BTK inhibitor or a pharmaceutically acceptable salt thereof, in the manufacture of a medicament for reducing or alleviating a symptom selected from the group consisting of night sweats, fatigue, pruritus, abdominal pain, pain under ribs, fullness, bone pain, and combinations thereof in a human subject with a MPN. In an embodiment, the BTK inhibitor is selected from Table 1.

### Symptoms of Myeloproliferative Neoplasms

In some embodiments, the disclosure relates to a method of reducing or alleviating a symptom associated with an MPN, such as night sweats, fatigue, pruritus, abdominal pain, pain under ribs, fullness, bone pain, or a combination thereof. In some embodiments, the disclosure relates to methods for reducing or alleviating a symptom selected from night sweats, fatigue, pruritus, abdominal pain, pain under ribs, fullness, bone pain, or a combination thereof in a human subject with an MPN comprising administering to the human subject an amount of a BTK inhibitor effective to reduce or alleviate the symptom. In some embodiments, the symptoms are associated with overproduction and release of pro-inflammatory cytokines by malignant cells. In some embodiments, the symptoms are associated with splenomegaly. In some embodiments, the symptoms are associated with overproduction and release of pro-inflammatory cytokines by malignant cells and with splenomegaly. In some embodiments, malignant cells of the human subject overproduce and release pro-inflammatory cytokines.

In some embodiments, the symptom persists for at least one day prior to administration of the BTK inhibitor, such as at least two days, at least three days, at least four days, at least five days, at least six days, or at least seven days prior to administration of the BTK inhibitor.

In some embodiments, the method further comprises assessing the severity of the symptom prior to administration of the BTK inhibitor. In some embodiments, the method further comprises assessing the severity of the symptom after administration of the BTK inhibitor. In some embodiments, severity of the symptom is determined by self-assessment. In some embodiments, severity of the symptom is determined by self-reporting.

In some embodiments, the severity of a symptom is assessed by assigning a numerical value (which may also be referred to as a score) to the severity of the symptom, for example, by assigning a value on a scale of zero to 10, wherein a value of zero indicates that a symptom is absent and a value of 10 indicates that a symptom's severity is the worst imaginable to the human subject. In some embodiments, a total symptom score is determined by summing the scores for each individual symptom. In some embodiments, the scores of two, three, four, five, six, seven, eight, nine, or ten symptoms are summed to obtain a total symptom score. In some embodiments, a symptom score or a total symptom score is obtained by reporting a score (for example, on a scale of zero to 10) in response to one, two, three, four, five, six, or seven of the following questions:

TABLE 2
Symptom Assessment Questionnaire
Question

(1) During the past 24 hours, how severe was your worse fatigue
(weariness, tiredness)?
(2) During the past 24 hours, how severe were your worst night
sweats (or feeling hot or flushed)?
(3) During the past 24 hours, how severe was your worst itching?
(4) During the past 24 hours, how severe was your worst abdominal
discomfort (feeling pressure or bloating)?
(5) During the past 24 hours, how severe was the worst pain under
your ribs on your left side?
(6) During the past 24 hours, what was the worst feeling of full-
ness you had after beginning to eat?
(7) During the past 24 hours, how severe was your worst bone pain
(not joint or arthritis pain)?

In some embodiments, the human subject is considered symptomatic and has a total symptom score assessed by the seven questions in Table 2, wherein each question is scored on a scale of zero to 10 with zero indicated the symptom is absent and 10 indicating the symptom is the worst imaginable, of at least 10, such as at least 12, at least 14, at least 15, at least 16, at least 18, at least 20, at least 22, at least 24, at least 25, at least 26, at least 28, at least 30, at least 32, at least 34, at least 35, at least 36, at least 38, at least 40, at least 42, at least 44, at least 45, at least 46, at least 48, and/or at least 50.

In some embodiments, a symptom is reduced if the symptom's score is reduced by at least 50%, such as at least 60%, at least 70%, at least 75%, at least 80%, and/or at least 90% compared to a baseline score for the symptom. In some embodiments, the baseline score for a symptom is the symptom's score before administering a BTK inhibitor as described herein. In some embodiments, the disclosure relates to a reduction in a total symptom score. In some embodiments, a total symptom score is reduced if the total symptom score is reduced by at least 50%, such as at least 60%, at least 70%, at least 75%, at least 80%, and/or at least 90% compared to a baseline total symptom score. In some embodiments, the baseline total symptom score is the total symptom score before administering a BTK inhibitor as described herein. In some embodiments, the total symptom score refers to an average of daily total symptom scores, such as a 7-day average of daily total symptom scores. In some embodiments, a reduction in a symptom and/or in a total symptom score is obtained after administering a BTK inhibitor as described herein for a period of time of at least 8 weeks, such as at least 12 weeks, at least 16 weeks, at least 20 weeks, at least 24 weeks, and/or at least 30 weeks.

In some embodiments, administering a BTK inhibitor as described herein improves the human subject's quality of life. In some embodiments, quality of life is assessed by the Patient Global Impression of Change (PGIC) assessment. PGIC is a seven point scale depicting a human subject's overall improvement, wherein the human subjects rate their change as “very much improved,” “much improved,” “minimally improved,” “no change,” “minimally worse,” “much worse,” or “very much worse.” In some embodiments, improved quality of life comprises a PGIC rating of “very much improved,” “much improved,” or “minimally improved.”

In some embodiments, the night sweats comprise repeated episodes of perspiration during sleep of the human subject. In some embodiments, the night sweats comprise repeated episodes of extreme perspiration during sleep of the human subject.

In some embodiments, the fatigue comprises unexplained and relapsing exhaustion of the human subject. In some embodiments, the fatigue comprises unexplained, persistent, and relapsing exhaustion of the human subject.

In some embodiments, the pruritus comprises itching of the skin of the human subject. In some embodiments, the pruritus comprises severe itching of the skin of the human subject.

In some embodiments, the abdominal pain occurs between the chest and pelvic regions and is acute. In some embodiments, the abdominal pain occurs between the chest and pelvic regions and is chronic.

In some embodiments, the pain under ribs occurs on the left side of the human subject and is acute. In some embodiments, the pain under ribs occurs on the left side of the human subject and is chronic.

In some embodiments, the fullness is associated with satiety in the human subject.

In some embodiments, the bone pain is characterized by tenderness, aching, or other discomfort in one or more bones of the human subject. In some embodiments, the bone pain is characterized by extreme tenderness, aching, or other discomfort in one or more bones of the human subject.

Combinations with JAK2 Inhibitors

In some embodiments, the disclosure relates to a method of reducing or alleviating a symptom selected from night sweats, fatigue, pruritus, abdominal pain, pain under ribs, fullness, bone pain, or a combination thereof by administering to a human subject in need thereof an effective amount of a BTK inhibitor in combination with a JAK2 inhibitor. The disclosure also relates to methods for reducing or alleviating a symptom selected from night sweats, fatigue, pruritus, abdominal pain, pain under ribs, fullness, bone pain, or a combination thereof by administering to a human subject with an MPN an effective amount of a BTK inhibitor in combination with a JAK2 inhibitor. The disclosure herein demonstrates that desirable therapeutic agents may be selected on the basis of BTK inhibition and JAK2 inhibition. Therefore, while not wishing to be bound to a particular mechanism of action, it is expected that BTK inhibition in combination with JAK2 inhibition alters one or more downstream signaling components (e.g., CXCR-4, CXCL12, VLA-4, VCAM1) to mobilize migration of CD34+ cells into the peripheral blood and will useful in the treatment of complications associated with MPNs, particularly in reducing or alleviating symptoms including, but not limited to, night sweats, fatigue, pruritus, abdominal pain, pain under ribs, fullness, bone pain, and combinations thereof.

The present disclosure thus relates to a method of reducing or alleviating symptoms associated with MPNs in a human subject suffering from an MPN comprising the step of administering to the human a BTK inhibitor compound or a pharmaceutically acceptable salt thereof in combination with a JAK2 inhibitor compound or a pharmaceutically acceptable salt thereof. In some embodiments, the JAK2 inhibitor is selected from ruxolitinib, fedratinib, pacritinib, momelotinib, jaktinib, ilginatinib, or a combination thereof. In some embodiments, the MPN is myelofibrosis (MF). In some embodiments, the MF is primary myelofibrosis, also known as chronic idiopathic myelofibrosis (cIMF). In an embodiment, the primary myelofibrosis is selected from the group consisting of prefibrotic/early stage PMF and overt fibrotic stage PMF. In some embodiments, the MF is secondary myelofibrosis. In some embodiments, the MF is myelofibrosis that develops secondary to polycythemia vera or essential thrombocythemia. In some embodiments, the MF is myelofibrosis that develops secondary to chronic myeloid leukemia (CML). In some embodiments, the MF is idiopathic myelofibrosis. In some embodiments, the MPN is a chronic myelogenous leukemia. In some embodiments, the MPN is acute myelogenous leukemia with antecedent MPN (also known as blast phase MPN (MPN-BP)). In some embodiments, the MPN is a chronic neutrophilic leukemia (CNL), a chronic eosinophilic leukemia, or a chronic myelomonocytic leukemia (CMML). In some embodiments, the MPN is an idiopathic systemic myeloproliferative neoplasm (MPN-U), a myelodysplastic syndrome (MDS), a systemic mast cell disease (SMCD), an atypical chronic myeloid leukemia (aCML), a juvenile myelomonocytic leukemia (JMML), a hypereosinophilic syndromes (HES), and a myelodysplastic/myeloproliferative neoplasm with ring sideroblasts and thrombocytosis (MDS/MPN-RS-T).

In an embodiment, the human is determined as hydroxyurea (HU) intolerant (unacceptable side effects). In an embodiment, the human subject is determined as hydroxyurea resistant (inadequate response).

In some embodiments, the human subject is additionally suffering from splenomegaly, hepatomegaly, or hepatosplenomegaly. In an embodiment, the human subject has splenomegaly, hepatomegaly, or hepatosplenomegaly and is phlebotomy-dependent. In an embodiment, the human subject is phlebotomy-dependent without splenomegaly, hepatomegaly, or hepatosplenomegaly.

In an embodiment, the BTK inhibitor is any of the compounds in Table 1 or a pharmaceutically acceptable salt thereof. In an embodiment, the JAK2 inhibitor is any of ruxolitinib, fedratinib, pacritinib, momelotinib, jaktinib, ilginatinib, a combination thereof, or a pharmaceutically acceptable salt thereof.

In some embodiments, the JAK2 inhibitor is administered once a day, two times per day, three times per day, four times per day, or five times per day. In an embodiment, the JAK2 inhibitor is administered in a dosage selected from the group consisting of 15 mg QD, 25 mg QD, 30 mg QD, 50 mg QD, 60 mg QD, 75 mg QD, 90 mg QD, 100 mg QD, 120 mg QD, 150 mg QD, 175 mg QD, 180 mg QD, 200 mg QD, 225 mg QD, 240 mg QD, 250 mg QD, 275 mg QD, 300 mg QD, 325 mg QD, 350 mg QD, 360 mg QD, 375 mg QD, 480 mg QD, 560 mg QD, 15 mg BID, 25 mg BID, 30 mg BID, 50 mg BID, 60 mg BID, 75 mg BID, 90 mg BID, 100 mg BID, 120 mg BID, 150 mg BID, 175 mg BID, 180 mg BID, 200 mg BID, 225 mg BID, 240 mg BID, 250 mg BID, 275 mg BID, 300 mg BID, 325 mg BID, 350 mg BID, 360 mg BID, 375 mg BID, and 480 mg BID. In an embodiment, the JAK2 inhibitor is administered to a human according to the Section herein titled Dosages and Dosing Regimens. In an embodiment, the BTK inhibitor is administered in a dosage selected from the group consisting of 15 mg QD, 25 mg QD, 30 mg QD, 50 mg QD, 60 mg QD, 75 mg QD, 90 mg QD, 100 mg QD, 120 mg QD, 150 mg QD, 175 mg QD, 180 mg QD, 200 mg QD, 225 mg QD, 240 mg QD, 250 mg QD, 275 mg QD, 300 mg QD, 325 mg QD, 350 mg QD, 360 mg QD, 375 mg QD, 480 mg QD, 560 mg QD, 15 mg BID, 25 mg BID, 30 mg BID, 50 mg BID, 60 mg BID, 75 mg BID, 90 mg BID, 100 mg BID, 120 mg BID, 150 mg BID, 175 mg BID, 180 mg BID, 200 mg BID, 225 mg BID, 240 mg BID, 250 mg BID, 275 mg BID, 300 mg BID, 325 mg BID, 350 mg BID, 360 mg BID, 375 mg BID, and 480 mg BID. In an embodiment, the BTK inhibitor is administered to a human according to the Section herein titled Dosages and Dosing Regimens. In some embodiments, the BTK inhibitor and the JAK2 inhibitor are administered simultaneously, sequentially or intermittently.

In some embodiments, the disclosure encompasses pharmaceutical combinations comprising: (a) a BTK inhibitor; (b) a JAK2 inhibitor; and (c) a pharmaceutically-acceptable excipient. In some embodiments, the combination provides a synergistic therapeutic effect compared to administration of the BTK inhibitor or the JAK2 inhibitor alone. In some embodiments, the combination sensitizes myelofibrosis to the BTK inhibitor. In some embodiments, the combination is in a combined dosage form. In some embodiments, the combination is in separate dosage forms.

In some embodiments, the disclosure encompasses use of a therapeutically effective amount of a combination comprising a BTK inhibitor and a JAK2 inhibitor for reducing or alleviating a symptom associated with MPNs such as night sweats, fatigue, pruritus, abdominal pain, pain under ribs, fullness, bone pain, and combinations thereof in a human subject with a MPN. In some embodiments, the combination provides a synergistic therapeutic effect compared to administration of the BTK inhibitor or the JAK2 inhibitor alone. In some embodiments, the combination sensitizes a malignant CD34+ myeloid cell to the BTK inhibitor. In some embodiments, the combination sensitizes a malignant CD34+ myeloid cell to the JAK2 inhibitor.

Combinations with MDM2 Inhibitors

In some embodiments, the disclosure relates to a method of reducing or alleviating a symptom selected from night sweats, fatigue, pruritus, abdominal pain, pain under ribs, fullness, bone pain, or a combination thereof by administering to a human subject in need thereof an effective amount of a BTK inhibitor in combination with an MDM2 inhibitor. The disclosure also relates to methods for reducing or alleviating a symptom selected from night sweats, fatigue, pruritus, abdominal pain, pain under ribs, fullness, bone pain, or a combination thereof by administering to a human subject with an MPN an effective amount of a BTK inhibitor in combination with an MDM2 inhibitor. The disclosure herein demonstrates that desirable therapeutic agents may be selected on the basis of BTK inhibition and MDM2 inhibition. Therefore, while not wishing to be bound to a particular mechanism of action, it is expected that BTK inhibition in combination with MDM2 inhibition alters one or more downstream signaling components (e.g., CXCR-4, CXCL12, VLA-4, VCAM1) to mobilize migration of CD34+ cells into the peripheral blood and will useful in the treatment of complications associated with MPNs, particularly in reducing or alleviating symptoms including, but not limited to, night sweats, fatigue, pruritus, abdominal pain, pain under ribs, fullness, bone pain, and combinations thereof.

The present disclosure thus relates to a method of reducing or alleviating symptoms associated with MPNs in a human subject suffering from an MPN comprising the step of administering to the human a BTK inhibitor compound or a pharmaceutically acceptable salt thereof in combination with an MDM2 inhibitor compound or a pharmaceutically acceptable salt thereof. In some embodiments, the MDM2 inhibitor is selected from any of the compounds in Table 3 or a pharmaceutically acceptable salt thereof.

TABLE 3
MDM2 Inhibitors

| No. | IUPAC Name |
|---|---|
| 1. | 2-[(3R,5R,6S)-5-(3-chlorophenyl)-6-(4-chlorophenyl)-3-methyl-1-[(2S)-3-methyl-1-propan-2- |
| | ylsulfonylbutan-2-yl]-2-oxopiperidin-3-yl]acetic acid |
| 2. | 4-[[(2R,3S,4R,5S)-3-(3-chloro-2-fluorophenyl)-4-(4-chloro-2-fluorophenyl)-4-cyano-5-(2,2- |
| | dimethylpropyl)pyrrolidine-2-carbonyl]amino]-3-methoxybenzoic acid |
| 3. | (5bS,6aS,7aS,8R,8aR,9aS,9bS,10aS,10bS)-8-hydroxy-8a-isopropyl-10b-methyl- |
| | 2,5,5b,6,6a,8,8a,9a,9b,10b-decahydrotris(oxireno) [2′,3′:4b,5; 2″,3″:6,7; 2″′,3″′:8a,9] |
| | phenanthro[1,2-c]furan-3(1H)-one |
| 4. | 4-[(4S,5R)-4,5-bis(4-chlorophenyl)-2-(4-methoxy-2-propan-2-yloxyphenyl)-4,5- |
| | dihydroimidazole-1-carbonyl]piperazin-2-one |
| 5. | (4S)-5-(5-chloro-1-methyl-2-oxopyridin-3-yl)-4-(4-chlorophenyl)-2-(2,4-dimethoxypyrimidin- |
| | 5-yl)-3-propan-2-yl-4H-pyrrolo[3,4-d]imidazol-6-one |
| 6. | [(4S,5R)-2-(4-tert-butyl-2-ethoxyphenyl)-4,5-bis(4-chlorophenyl)-4,5-dimethylimidazol-1-yl]- |
| | [4-(3-methylsulfonylpropyl)piperazin-1-yl]methanone |
| 7. | (1S)-1-(4-chlorophenyl)-6-methoxy-2-[4-[methyl-[[4-(4-methyl-3-oxopiperazin-1- |
| | yl)cyclohexyl]methyl]amino]phenyl]-7-propan-2-yloxy-1,4-dihydroisoquinolin-3-one |
| 8. | 4-[4,5-bis(4-chlorophenyl)-2-(4-methoxy-2-propan-2-yloxyphenyl)-4,5-dihydroimidazole-1- |
| | carbonyl]piperazin-2-one |
| 9. | methyl 2-[2-chloro-6-ethoxy-4-[(3-methyl-5-oxo-1-phenylpyrazol-4- |
| | ylidene)methyl]phenoxy]acetate |
| 10. | (2′R,3R,3′S,5′S)-6-chloro-3′-(3-chloro-2-fluorophenyl)-5′-(2,2-dimethylpropyl)-N-(4- |
| | hydroxycyclohexyl)-2-oxospiro[1H-indole-3,4′-pyrrolidine]-2′-carboxamide |
| 11. | (2′R,3S,3′S,5′R)-6-chloro-3′-(3-chloro-2-fluorophenyl)-5′-(2,2-dimethylpropyl)-N-(4- |
| | hydroxycyclohexyl)-2-oxospiro[1H-indole-3,4′-pyrrolidine]-2′-carboxamide |
| 12. | 4-[8-[(3,4-dimethylphenyl)sulfamoyl]-3a,4,5,9b-tetrahydro-3H-cyclopenta[c]quinolin-4- |
| | yl]benzoic acid |
| 13. | ethyl 3-[2-(tert-butylamino)-1-[(4-chlorophenyl)methyl-formylamino]-2-oxoethyl]-6-chloro- |
| | 1H-indole-2-carboxylate |
| 14. | (2′R,3R,3′S,5′S)-N-(4-carbamoyl-2-methoxyphenyl)-6-chloro-3′-(3-chloro-2-fluorophenyl)-5′- |
| | (2,2-dimethylpropyl)-2-oxospiro[1H-indole-3,4′-pyrrolidine]-2′-carboxamide |
| 15. | 4-[(4R,5S)-4,5-bis(4-chlorophenyl)-2-(4-methoxy-2-propan-2-yloxyphenyl)-4,5- |
| | dihydroimidazole-1-carbonyl]piperazin-2-one |
| 16. | 1-N-[2-(1H-indol-3-yl)ethyl]-4-N-pyridin-4-ylbenzene-1,4-diamine |
| 17. | (E)-1-(4-methylpiperazin-1-yl)-3-(5-nitrofuran-2-yl)prop-2-en-1-one |
| 18. | 2-[4-[(4S,5R)-2-(4-tert-butyl-2-ethoxyphenyl)-4,5-bis(4-chlorophenyl)-4,5- |
| | dimethylimidazole-1-carbonyl]piperazin-1-yl]-1-morpholin-4-ylethanone |
| 19. | (1R)-1-(4-chlorophenyl)-6-methoxy-2-[4-[methyl-[[4-(4-methyl-3-oxopiperazin-1- |
| | yl)cyclohexyl]methyl]amino]phenyl]-7-propan-2-yloxy-1,4-dihydroisoquinolin-3-one |
| 20. | 4-amino-1-[(2R,3S,4S,5R)-3,4-dihydroxy-5-(hydroxymethyl)oxolan-2-yl]pyrimidin-2-one |
| 21. | (3′R,4′S,5′R)-N-((3R,6S)-6-carbamoyltetrahydro-2H-pyran-3-yl)-6″-chloro-4′-(2-chloro-3- |
| | fluoropyridin-4-yl)-4,4-dimethyl-2″-oxodispiro[cyclohexane-1,2′-pyrrolidine-3′,3″-indoline]- |
| | 5′-carboxamide |
| 22. | (3′R,4′S,5′R)-N-((3R,6S)-6-carbamoyltetrahydro-2H-pyran-3-yl)-6″-chloro-4′-(2-chloro-3- |
| | fluoropyridin-4-yl)-4,4-dimethyl-2″-oxodispiro[cyclohexane-1,2′-pyrrolidine-3′,3″-indoline]- |
| | 5′-carboxamide 4-methylbenzenesulfonate |
| 23. | 4-((3′R,4′S,5′R)-6″-Chloro-4′-(3-chloro-2-fluorophenyl)-1′-ethyl-2″-oxodispiro[cyclohexane- |
| | 1,2′-pyrrolidine-3′,3″-indoline]-5′-carboxamido)bicyclo[2.2.2]octane-1-carboxylic Acid |
| 24. | 4-((3′R,4′S,5′R)-6″-chloro-4′-(3-chloro-2-fluorophenyl)-2″-oxodispiro[cyclohexane-1,2′- |
| | pyrrolidine-3′,3″-indoline]-5′-carboxamido)benzoic acid |

In some embodiments, the MPN is myelofibrosis (MF). In some embodiments, the MF is primary myelofibrosis, also known as chronic idiopathic myelofibrosis (cIMF). In an embodiment, the primary myelofibrosis is selected from the group consisting of prefibrotic/early stage PMF and overt fibrotic stage PMF. In some embodiments, the MF is secondary myelofibrosis. In some embodiments, the MF is myelofibrosis that develops secondary to polycythemia vera or essential thrombocythemia. In some embodiments, the MF is myelofibrosis that develops secondary to chronic myeloid leukemia (CML). In some embodiments, the MF is idiopathic myelofibrosis. In some embodiments, the MPN is chronic myelogenous leukemia. In some embodiments, the MPN is acute myelogenous leukemia with antecedent MPN (also known as blast phase MPN (MPN-BP)). In some embodiments, the MPN is a chronic neutrophilic leukemia (CNL), a chronic eosinophilic leukemia, or a chronic myelomonocytic leukemia (CMML). In some embodiments, the MPN is an idiopathic systemic mastocystosis (SM), a chronic eosinophilic leukemia-not otherwise specified (CEL-NOS), an unclassified myeloproliferative neoplasm (MPN-U), a myelodysplastic syndrome (MDS), a systemic mast cell disease (SMCD), an atypical chronic myeloid leukemia (aCML), a juvenile myelomonocytic leukemia (JMML), a hypereosinophilic syndromes (HES), and a myelodysplastic/myeloproliferative neoplasm with ring sideroblasts and thrombocytosis (MDS/MPN-RS-T).

In an embodiment, the human is determined as hydroxyurea (HU) intolerant (unacceptable side effects). In an embodiment, the human subject is determined as hydroxyurea resistant (inadequate response).

In some embodiments, the human subject is additionally suffering from splenomegaly, hepatomegaly, or hepatosplenomegaly. In an embodiment, the human subject has splenomegaly, hepatomegaly, or hepatosplenomegaly and is phlebotomy-dependent. In an embodiment, the human subject is phlebotomy-dependent without splenomegaly, hepatomegaly, or hepatosplenomegaly.

In an embodiment, the BTK inhibitor is any of the compounds in Table 1 or a pharmaceutically acceptable salt thereof. In an embodiment, the MDM2 inhibitor is any of the compounds in Table 3, a combination thereof, or a pharmaceutically acceptable salt thereof.

In some embodiments, the MDM2 inhibitor is administered once a day, two times per day, three times per day, four times per day, or five times per day. In an embodiment, the MDM2 inhibitor is administered in a dosage selected from the group consisting of 15 mg QD, 25 mg QD, 30 mg QD, 50 mg QD, 60 mg QD, 75 mg QD, 90 mg QD, 100 mg QD, 120 mg QD, 150 mg QD, 175 mg QD, 180 mg QD, 200 mg QD, 225 mg QD, 240 mg QD, 250 mg QD, 275 mg QD, 300 mg QD, 325 mg QD, 350 mg QD, 360 mg QD, 375 mg QD, 480 mg QD, 560 mg QD, 15 mg BID, 25 mg BID, 30 mg BID, 50 mg BID, 60 mg BID, 75 mg BID, 90 mg BID, 100 mg BID, 120 mg BID, 150 mg BID, 175 mg BID, 180 mg BID, 200 mg BID, 225 mg BID, 240 mg BID, 250 mg BID, 275 mg BID, 300 mg BID, 325 mg BID, 350 mg BID, 360 mg BID, 375 mg BID, and 480 mg BID. In an embodiment, the MDM2 inhibitor is administered to a human according to the Section herein titled Dosages and Dosing Regimens. In an embodiment, the BTK inhibitor is administered in a dosage selected from the group consisting of 15 mg QD, 25 mg QD, 30 mg QD, 50 mg QD, 60 mg QD, 75 mg QD, 90 mg QD, 100 mg QD, 120 mg QD, 150 mg QD, 175 mg QD, 180 mg QD, 200 mg QD, 225 mg QD, 240 mg QD, 250 mg QD, 275 mg QD, 300 mg QD, 325 mg QD, 350 mg QD, 360 mg QD, 375 mg QD, 480 mg QD, 560 mg QD, 15 mg BID, 25 mg BID, 30 mg BID, 50 mg BID, 60 mg BID, 75 mg BID, 90 mg BID, 100 mg BID, 120 mg BID, 150 mg BID, 175 mg BID, 180 mg BID, 200 mg BID, 225 mg BID, 240 mg BID, 250 mg BID, 275 mg BID, 300 mg BID, 325 mg BID, 350 mg BID, 360 mg BID, 375 mg BID, and 480 mg BID. In an embodiment, the BTK inhibitor is administered to a human according to the Section herein titled Dosages and Dosing Regimens. In some embodiments, the BTK inhibitor and the MDM2 inhibitor are administered simultaneously, sequentially or intermittently.

In some embodiments, the disclosure encompasses pharmaceutical combinations comprising: (a) a BTK inhibitor; (b) an MDM2 inhibitor; and (c) a pharmaceutically-acceptable excipient. In some embodiments, the combination provides a synergistic therapeutic effect compared to administration of the BTK inhibitor or the MDM2 inhibitor alone. In some embodiments, the combination sensitizes myelofibrosis to the BTK inhibitor. In some embodiments, the combination is in a combined dosage form. In some embodiments, the combination is in separate dosage forms.

In some embodiments, the disclosure encompasses use of a therapeutically effective amount of a combination comprising a BTK inhibitor and an MDM2 inhibitor for reducing or alleviating a symptom associated with MPNs such as night sweats, fatigue, pruritus, abdominal pain, pain under ribs, fullness, bone pain, and combinations thereof in a human subject with a MPN. In some embodiments, the combination provides a synergistic therapeutic effect compared to administration of the BTK inhibitor or the MDM2 inhibitor alone. In some embodiments, the combination sensitizes a malignant CD34+ myeloid cell to the BTK inhibitor. In some embodiments, the combination sensitizes a malignant CD34+ myeloid cell to the MDM2 inhibitor.

### Pharmaceutical Compositions

In some embodiments, the disclosure provides pharmaceutical compositions comprising a BTK inhibitor compound or a pharmaceutically acceptable salt thereof for reducing or alleviating a symptom selected from night sweats, fatigue, pruritus, abdominal pain, pain under ribs, fullness, bone pain, or a combination thereof. In some embodiments, the disclosure provides pharmaceutical compositions comprising a BTK inhibitor compound or a pharmaceutically acceptable salt thereof for reducing or alleviating a symptom selected from night sweats, fatigue, pruritus, abdominal pain, pain under ribs, fullness, bone pain, or a combination thereof in a human subject with an MPN. In an embodiment, the MPN is selected from myelofibrosis, primary myelofibrosis, prefibrotic/early stage PMF, overt fibrotic stage PMF, secondary myelofibrosis, myelofibrosis that develops secondary to polycythemia vera or essential thrombocythemia, myelofibrosis that develops secondary to chronic myeloid leukemia (CML), idiopathic myelofibrosis, chronic myelogenous leukemia, acute myelogenous leukemia with antecedent MPN (also known as blast phase MPN (MPN-BP)), a chronic neutrophilic leukemia (CNL), a chronic eosinophilic leukemia, a chronic myelomonocytic leukemia (CMML), an idiopathic systemic mastocystosis (SM), a chronic eosinophilic leukemia-not otherwise specified (CEL-NOS), an unclassified myeloproliferative neoplasm (MPN-U), a myelodysplastic syndrome (MDS), a systemic mast cell disease (SMCD), an atypical chronic myeloid leukemia (aCML), a juvenile myelomonocytic leukemia (JMML), a hypereosinophilic syndromes (HES), or a myelodysplastic/myeloproliferative neoplasm with ring sideroblasts and thrombocytosis (MDS/MPN-RS-T).

The pharmaceutical compositions are typically formulated to provide a therapeutically effective amount of a BTK inhibitor compound or a pharmaceutically acceptable salt thereof. Where desired, the pharmaceutical compositions contain a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants. Where desired, other ingredients in addition to the BTK inhibitor or a pharmaceutically acceptable salt thereof may be mixed into a preparation or both components may be formulated into separate preparations for use in combination separately or at the same time.

In selected embodiments, the concentration of the BTK inhibitor or a pharmaceutically acceptable salt thereof provided in the pharmaceutical compositions of the disclosure is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v.

In selected embodiments, the concentration of the BTK inhibitor or a pharmaceutically acceptable salt thereof provided in the pharmaceutical compositions of the disclosure is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v.

In selected embodiments, the concentration of the BTK inhibitor or a pharmaceutically acceptable salt thereof is independently in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12% or approximately 1% to approximately 10% w/w, w/v or v/v.

In selected embodiments, the concentration of the BTK inhibitor or a pharmaceutically acceptable salt thereof is independently in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In selected embodiments, the amount of the BTK inhibitor or a pharmaceutically acceptable salt thereof is independently equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g or 0.0001 g.

In selected embodiments, the amount of the BTK inhibitor or a pharmaceutically acceptable salt thereof is independently more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g or 10 g.

The BTK inhibitor compounds in Table 1 and pharmaceutically acceptable salts thereof are effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting exemplary pharmaceutical compositions and methods for preparing the same.

### Pharmaceutical Compositions for Oral Administration

In selected embodiments, the disclosure provides a pharmaceutical composition for oral administration comprising a BTK inhibitor compound or a pharmaceutically acceptable salt thereof, and a pharmaceutical excipient suitable for oral administration.

In selected embodiments, the disclosure provides a solid pharmaceutical composition for oral administration containing: (i) an effective amount of the BTK inhibitor or a pharmaceutically acceptable salt thereof, in combination and (ii) a pharmaceutical excipient suitable for oral administration. In selected embodiments, the composition further contains (iii) an effective amount of at least one additional active ingredient.

In selected embodiments, the pharmaceutical composition may be a liquid pharmaceutical composition suitable for oral consumption. Pharmaceutical compositions of the disclosure suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

The BTK inhibitor or a pharmaceutically acceptable salt thereof can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use, such as for compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, epsilon-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Examples may include, but are not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In selected embodiments, the disclosure provides a pharmaceutical composition for injection comprising a BTK inhibitor compound or a pharmaceutically acceptable salt thereof, and a pharmaceutical excipient suitable for injection. Components and amounts of agents in the compositions are as described herein.

The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating the BTK inhibitor or a pharmaceutically acceptable salt thereof in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Administration of the BTK inhibitor or a pharmaceutically acceptable salt thereof or pharmaceutical composition of these compounds can be effected by any method that enables delivery of the compounds to the site of action. These methods include oral routes, intraduodenal routes, parenteral injection (including intravenous, intra-arterial, subcutaneous, intramuscular, intravascular, intraperitoneal or infusion), topical (e.g., transdermal application), rectal administration, via local delivery by catheter or stent or through inhalation. The compounds can also be administered intraadiposally or intrathecally.

Exemplary parenteral administration forms include solutions or suspensions of active compound in sterile aqueous solutions, for example, aqueous propylene glycol or dextrose solutions. Such dosage forms can be suitably buffered, if desired.

The disclosure also provides kits. The kits include a pharmaceutical composition comprising a BTK inhibitor compound or a pharmaceutically acceptable salt thereof, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer. In an embodiment, the disclosure provides a kit comprising a pharmaceutical composition comprising the BTK inhibitor or a pharmaceutically acceptable salt thereof for use in reducing or alleviating a symptom selected of an MPN (e.g., myelofibrosis) such as night sweats, fatigue, pruritus, abdominal pain, pain under ribs, fullness, bone pain, or a combination thereof as described herein.

### Dosages and Dosing Regimens

The amount of a BTK inhibitor or a pharmaceutically acceptable salt thereof administered will be dependent on the human being treated, the severity of the disorder or condition, the rate of administration, the disposition of the compounds and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day.

In some embodiments, a BTK inhibitor or a pharmaceutically acceptable salt thereof is administered in a single dose. Mutiple daily doses are also embodied, for example, twice daily. Typically, such administration will be oral. However, other routes may be used as appropriate.

In some embodiments, a BTK inhibitor or a pharmaceutically acceptable salt thereof is administered in multiple doses for reducing or alleviating a symptom associated with an MPN (e.g., myelofibrosis) selected from night sweats, fatigue, pruritus, abdominal pain, pain under ribs, fullness, bone pain, or a combination thereof. In an embodiment, a BTK inhibitor or a pharmaceutically acceptable salt thereof is administered in multiple doses. In an embodiment, dosing may be once, twice, three times, four times, five times, six times, or more than six times per day. In an embodiment, dosing may be selected from the group consisting of once a day, twice a day, three times a day, four times a day, five times a day, six times a day, once every other day, once weekly, twice weekly, three times weekly, four times weekly, biweekly, and monthly. In other embodiments, a BTK inhibitor or a pharmaceutically acceptable salt thereof is administered about once per day to about six times per day, such as once per day to about four times per day. In some embodiments a BTK inhibitor or a pharmaceutically acceptable salt thereof is administered once daily, while in other embodiments a BTK inhibitor or a pharmaceutically acceptable salt thereof is administered twice daily, and in other embodiments a BTK inhibitor or a pharmaceutically acceptable salt thereof is administered three times daily. In some embodiments a BTK inhibitor or a pharmaceutically acceptable salt thereof is administered three times a week, including every Monday, Wednesday, and Friday.

Administration of a BTK inhibitor or a pharmaceutically acceptable salt thereof may continue as long as necessary. In some embodiments, a BTK inhibitor or a pharmaceutically acceptable salt thereof is administered for more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more days. In some embodiments, a BTK inhibitor or a pharmaceutically acceptable salt thereof is administered for less than 28, 14, 7, 6, 5, 4, 3, 2, or 1 day. In some embodiments, a BTK inhibitor or a pharmaceutically acceptable salt thereof is administered for about 14 days, about 21 days, about 28 days, about 35 days, about 42 days, about 49 days, or about 56 days. In some embodiments, a BTK inhibitor or a pharmaceutically acceptable salt thereof is administered chronically on an ongoing basis—e.g., for the treatment of chronic effects. In another embodiment the administration of a BTK inhibitor or a pharmaceutically acceptable salt thereof continues for less than about 7 days. In yet another embodiment the administration continues for more than about 6, 10, 14, 28 days, two months, three months, four months, five months, six months, seven months, eight months, nine months, ten months, eleven months or one year. In some embodiments, the administration continues for more than about one year, two years, three years, four years, or five years. In some embodiments, continuous dosing is achieved and maintained as long as necessary.

In some embodiments, an effective dosage of a BTK inhibitor or a pharmaceutically acceptable salt thereof is in the range of about 1 mg to about 600 mg, about 10 mg to about 500 mg, about 20 mg to about 450 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of a BTK inhibitor or a pharmaceutically acceptable salt thereof is about 15 mg, about 25 mg, about 30 mg, about 50 mg, about 50 mg, about 75 mg, about 90 mg, about 100 mg, about 120 mg, about 125 mg, about 150 mg, about 175 mg, about 180 mg, about 200 mg, about 225 mg, about 240 mg, about 250 mg, about 275 mg, about 300 mg, about 325 mg, about 350 mg, about 360 mg, about 375 mg, about 400 mg, about 425 mg, about 450 mg, about 475 mg, about 480 mg, or about 500 mg. In some embodiments, an effective dosage of a BTK inhibitor or a pharmaceutically acceptable salt thereof is 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg.

In some embodiments, an effective dosage of a BTK inhibitor or a pharmaceutically acceptable salt thereof is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of a BTK inhibitor or a pharmaceutically acceptable salt thereof is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, a BTK inhibitor or a pharmaceutically acceptable salt thereof is administered at a dosage of 10 to 500 mg BID, including a dosage of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg BID.

In some embodiments, a BTK inhibitor or a pharmaceutically acceptable salt thereof is administered at a dosage of 10 to 600 mg QD, including a dosage of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg QD.

An effective amount of a BTK inhibitor or a pharmaceutically acceptable salt thereof may be administered in either single or multiple doses by any of the accepted modes of administration of agents having similar utilities, including buccal, sublingual, and transdermal routes, by intra-arterial injection, intravenously, parenterally, intramuscularly, subcutaneously or orally.

In some embodiments, a BTK inhibitor or a pharmaceutically acceptable salt thereof is administered to a subject intermittently, known as intermittent administration. By “intermittent administration,” it is meant a period of administration of a therapeutically effective dose of a BTK inhibitor or a pharmaceutically acceptable salt thereof, followed by a time period of discontinuance, which is then followed by another administration period and so on. In each administration period, the dosing frequency can be independently select from three times daily, twice daily, daily, once weekly, twice weekly, three times weekly, four times weekly, five times weekly, six times weekly or monthly. In an embodiment, the BTK inhibitor is a compound selected from Table 1 or a pharmaceutically acceptable salt thereof.

By “period of discontinuance” or “discontinuance period” or “rest period”, it is meant to the length of time when discontinuing of the administration of a BTK inhibitor or a pharmaceutically acceptable salt thereof. The time period of discontinuance may be longer or shorter than the administration period or the same as the administration period. During the discontinuance period, other therapeutic agents other than a BTK inhibitor or a pharmaceutically acceptable salt thereof may be administered. The discontinuance period may be necessary to alleviate any toxic effects associated with a particular BTK inhibitor compound.

In an embodiment, a BTK inhibitor or a pharmaceutically acceptable salt thereof is administered to a human subject in need thereof for reducing or alleviating a symptom associated with an MPN (e.g., myelofibrosis) such as night sweats, fatigue, pruritus, abdominal pain, pain under ribs, fullness, bone pain, or a combination thereof for a first administration period, then followed by a discontinuance period, then followed by a second administration period, and so on. The first administration period, the second administration period, and the discontinuance period are independently selected from the group consisting of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, one month, five weeks, six weeks, seven weeks, two months, nine weeks, ten weeks, elven weeks, three months, thirteen weeks, fourteen weeks, fifteen weeks, four months, and more days, in which a BTK inhibitor or a pharmaceutically acceptable salt thereof is administered to a subject three times daily, twice daily, daily, once weekly, twice weekly, three times weekly, four times weekly, five times weekly, six times weekly or monthly. In an embodiment, the first administration period is same length as the second administration period. In an embodiment, the first administration period is shorter than the second administration period. In an embodiment, the first administration period is longer than the second administration period. In an embodiment, the first administration period and the second administration period are about one week, in which a BTK inhibitor or a pharmaceutically acceptable salt thereof is administered to a subject daily; and the discontinuance period is about two weeks. In an embodiment, the first administration period and the second administration period are about three weeks, in which a BTK inhibitor or a pharmaceutically acceptable salt thereof is administered to a subject daily; and the discontinuance period is about two weeks. In an embodiment, the first administration period and the second administration period are about three weeks, in which a BTK inhibitor or a pharmaceutically acceptable salt thereof is administered to a subject weekly; and the discontinuance period is about two weeks. In an embodiment, the first administration period and the second administration period are about four weeks, in which a BTK inhibitor or a pharmaceutically acceptable salt thereof is administered to a subject daily; and the discontinuance period is about two weeks. In an embodiment, the first administration period and the second administration period are about four weeks, in which a BTK inhibitor or a pharmaceutically acceptable salt thereof is administered to a subject weekly; and the discontinuance period is about two weeks. In an embodiment, the BTK inhibitor is a compound selected from Table 1 or a pharmaceutically acceptable salt thereof.

In an embodiment, a BTK inhibitor or a pharmaceutically acceptable salt thereof is administered to a subject in need thereof for reducing or alleviating a symptom associated with an MPN (e.g., myelofibrosis) such as night sweats, fatigue, pruritus, abdominal pain, pain under ribs, fullness, bone pain, or a combination thereof for a period selected from 3 weeks, 6 weeks, 9 weeks, 12 weeks, 15 weeks, 18 weeks, 21 weeks, 24 weeks, 27 weeks, 30 weeks, 33 weeks, 36 weeks, 39 weeks, 42 weeks, 45 weeks, 48 weeks, 51 weeks, 54 weeks, 57 weeks, 60 weeks, 63 weeks, 66 weeks, 69 weeks, 72 weeks, 75 weeks, 78 weeks, 81 weeks, 84 weeks, 87 weeks, 90 weeks, 93 weeks, 96 weeks, 99 weeks, 102 weeks, 105 weeks, 108 weeks, 111 weeks, 114 weeks, 117 weeks, 120 weeks, 123 weeks, 126 weeks, 129 weeks, 132 weeks, 135 weeks, 138 weeks, 141 weeks, 144 weeks, 147 weeks, 150 weeks, 153 weeks, and 156 weeks, wherein the BTK inhibitor is selected from any of the compounds in Table 1 or a pharmaceutically acceptable salt thereof. In an embodiment, the BTK inhibitor is orally administered at a dose of 100 mg twice a day.

1. A method of reducing or alleviating a symptom selected from the group consisting of night sweats, fatigue, pruritus, abdominal pain, pain under ribs, fullness, bone pain, and combinations thereof in a human subject with a myeloproliferative neoplasm (MPN) comprising administering to the human subject an amount of a Bruton's Tyrosine Kinase (BTK) inhibitor effective to reduce or alleviate the symptom.

2. The method of claim 1, wherein the MPN is primary myelofibrosis.

3. The method of claim 1, wherein the MPN is post-polycythemia vera myelofibrosis.

4. The method of claim 1, wherein the MPN is post-essential thrombocythemia myelofibrosis.

5. The method of claim 1, wherein the MPN is chronic myelogenous leukemia.

6. The method of claim 1, wherein the MPN is chronic neutrophilic leukemia.

7. The method of claim 1, wherein the MPN is chronic eosinophilic leukemia.

8. The method of claim 1, wherein the MPN is acute myelogenous leukemia with antecedent MPN (also known as blast phase MPN (MPN-BP)).

9. The method of claim 1, wherein the MPN is chronic myelomonocytic leukemia.

10. The method of claim 1, wherein the human subject is suffering from splenomegaly, hepatomegaly, or hepatosplenomegaly.

11. The method of claim 1, wherein the BTK inhibitor is administered in combination with a JAK2 inhibitor.

12. The method of claim 11, wherein the JAK2 inhibitor is ruxolitinib, fedratinib, pacritinib, momelotinib, jaktinib, or ilginatinib.

13. The method of claim 1, wherein the BTK inhibitor is administered in combination with a MDM2 inhibitor.

14. The method of claim 1, wherein the MPN is a JAK2-V617F myeloproliferative neoplasm.

15. The method of claim 1, wherein the symptom persists for at least one day prior to administration of the BTK inhibitor, optionally at least two days, at least three days, at least four days, at least five days, at least six days, or at least seven days prior to administration of the BTK inhibitor.

16. The method of claim 1, wherein the method further comprises assessing the severity of the symptom prior to administration of the BTK inhibitor.

17. The method of claim 1, wherein the method further comprises assessing the severity of the symptom after administration of the BTK inhibitor.

18. The method of claim 1, wherein severity of the symptom is determined by self-assessment.

19. The method of claim 1, wherein severity of the symptom is determined by self-reporting.

20. The method of claim 1, wherein the night sweats comprise repeated episodes of perspiration during sleep of the human subject, such as repeated episodes of extreme perspiration during sleep of the human subject.

21. The method of claim 1, wherein the fatigue comprises unexplained and relapsing exhaustion of the human subject, such as unexplained, persistent, and relapsing exhaustion of the human subject.

22. The method of claim 1, wherein the pruritus comprises itching of the skin of the human subject, such as severe itching of the skin of the human subject.

23. The method of claim 1, wherein the abdominal pain occurs between the chest and pelvic regions and is acute or chronic.

24. The method of claim 1, wherein the pain under ribs occurs on the left side of the human subject and is acute or chronic.

25. The method of claim 1, wherein the fullness is associated with satiety in the human subject.

26. The method of claim 1, wherein the bone pain is characterized by tenderness, aching, or other discomfort in one or more bones of the human subject, such as extreme tenderness, aching, or other discomfort in one or more bones of the human subject.

27. The method of claim 1, wherein the BTK inhibitor is selected from Table 1 of the specification or a pharmaceutically acceptable salt thereof.

28. The method of claim 1, wherein the BTK inhibitor is I-(4-(((6-amino-5-(4-phenoxyphenyl)pyrimidin-4-yl)amino)methyl)-4-fluoropiperidin-1-yl)prop-2-en-I-one or a pharmaceutically acceptable salt thereof.
